SEQUENCE LISTING (30 sequences)

SEQ ID NO: 1 - protein, 124 aa, Artificial Sequence
  Synthetic
MNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKN

SEQ ID NO: 2 - protein, 670 aa, Artificial Sequence
  Synthetic
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 3 - protein, 519 aa, Artificial Sequence
  Synthetic
ALYDSSSYVYYYQSAFRPPDGWHLHGGAYAVVNISSESNNAGSSSGCTVGIIHGGRVVNASSIAMTAPSSGMAWSSSQFCTAYCNFSDTTVFVTHCYKHGGCPITGMLQQHSIRVSAMKNGQLFYNLTVSVAKYPTFKSFQCVNNLTSVYLNGDLVYTSNETTDVTSAGVYFKAGGPITYKVMREVRALAYFVNGTAQDVILCDGSPRGLLACQYNTGNFSDGFYPFTNSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPLLCKGVYSGELDHNFECGLLVYVTKSGGSRIQTATEPPVITQHNYNNITLNTCVDYNIYGRTGQGFITNVTDSAVSYNYLADAGLAILDTSGSIDIFVVQSEYGLNYYKVNPCEDVNQQFVVSGGKLVGILTSRNETGSQLLENQFYIKITNGTRRFRR

SEQ ID NO: 4 - protein, 130 aa, Artificial Sequence
  Synthetic
NNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGS

SEQ ID NO: 5 - protein, 112 aa, Artificial Sequence
  Synthetic
AAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKT

SEQ ID NO: 6 - protein, 134 aa, Artificial Sequence
  Synthetic
VGSSGNASWFQALKAKKLNSPPPKFEGSGVPDNENLKLSQQHGYWRRQARYKPGKGGKKSVPDAWYFYYTGTGPAADLNWGDSQDGIVWVSAKGADTKSRSNQGTRDPDKFDQYPLRFSDGGPDGNFRWDFIPI

SEQ ID NO: 7 - protein, 109 aa, Artificial Sequence
  Synthetic
KADEMAHRRYCKRTIPPGYKVDQVFGPRTKGKEGNFGDDKMNEEGIKDGRVIAMLNLVPSSHACLFGSRVTPKLQPDGLHLRFEFTTVVSRDDPQFDNYVKICDQCVDG

SEQ ID NO: 8 - protein, 802 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 9 - protein, 260 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGS

SEQ ID NO: 10 - protein, 242 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKT

SEQ ID NO: 11 - protein, 649 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGALYDSSSYVYYYQSAFRPPDGWHLHGGAYAVVNISSESNNAGSSSGCTVGIIHGGRVVNASSIAMTAPSSGMAWSSSQFCTAYCNFSDTTVFVTHCYKHGGCPITGMLQQHSIRVSAMKNGQLFYNLTVSVAKYPTFKSFQCVNNLTSVYLNGDLVYTSNETTDVTSAGVYFKAGGPITYKVMREVRALAYFVNGTAQDVILCDGSPRGLLACQYNTGNFSDGFYPFTNSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPLLCKGVYSGELDHNFECGLLVYVTKSGGSRIQTATEPPVITQHNYNNITLNTCVDYNIYGRTGQGFITNVTDSAVSYNYLADAGLAILDTSGSIDIFVVQSEYGLNYYKVNPCEDVNQQFVVSGGKLVGILTSRNETGSQLLENQFYIKITNGTRRFRR

SEQ ID NO: 12 - protein, 264 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGVGSSGNASWFQALKAKKLNSPPPKFEGSGVPDNENLKLSQQHGYWRRQARYKPGKGGKKSVPDAWYFYYTGTGPAADLNWGDSQDGIVWVSAKGADTKSRSNQGTRDPDKFDQYPLRFSDGGPDGNFRWDFIPI

SEQ ID NO: 13 - protein, 239 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGKADEMAHRRYCKRTIPPGYKVDQVFGPRTKGKEGNFGDDKMNEEGIKDGRVIAMLNLVPSSHACLFGSRVTPKLQPDGLHLRFEFTTVVSRDDPQFDNYVKICDQCVDG

SEQ ID NO: 14 - DNA, 375 bp, Artificial Sequence
  Synthetic
atgggcaacaaggtgaaatgctacgttctgttcaccgcgctgctgagcagcctgtatgcgcacggtgcgccgcagaccattaccgagctgtgcagcgaataccgtaacacccaaatctataccattaacgacaagatcctgagctacaccgagagcatggcgggcaagcgtgaaatggtgatcattaccttcaaaagcggtgaaacctttcaggtggaagttccgggcagccagcacatcgacagccaaaagaaagcgattgagcgtatgaaagataccctgcgtatcacctatctgaccgaaaccaagattgacaaactgtgcgtgtggaacaacaagaccccgaacagcatcgcggcgattagcatgaaaaac

SEQ ID NO: 15 - DNA, 780 bp, Artificial Sequence
  Synthetic
atgggcaacaaggtgaaatgctacgttctgttcaccgcgctgctgagcagcctgtatgcgcacggtgcgccgcagaccattaccgagctgtgcagcgaataccgtaacacccaaatctataccattaacgacaagatcctgagctacaccgagagcatggcgggcaagcgtgaaatggtgatcattaccttcaaaagcggtgaaacctttcaggtggaagttccgggcagccagcacatcgacagccaaaagaaagcgattgagcgtatgaaagataccctgcgtatcacctatctgaccgaaaccaagattgacaaactgtgcgtgtggaacaacaagaccccgaacagcatcgcggcgattagcatgaaaaacggtggcagcggtggcaacaacaccgcgagctggttcaccgcgctgacccagcacggtaaagaggacctgaaatttccgcgtggtcaaggcgttccgatcaacaccaacagcagcccggacgatcaaattggttactatcgtcgtgcgacccgtcgtatccgtggtggcgacggcaagatgaaggacctgagcccgcgttggtacttctactacctgggcaccggtccggaggcgggtctgccgtatggcgcgaacaaggacggtatcatttgggtggcgaccgaaggcgcgctgaacaccccgaaagatcacattggcacccgtaacccggcgaacaacgcggcgatcgttctgcaactgccgcaaggcaccaccctgccgaagggtttttacgcggaaggcagc

SEQ ID NO: 16 - DNA, 726 bp, Artificial Sequence
  Synthetic
atgggcaacaaggtgaaatgctacgttctgttcaccgcgctgctgagcagcctgtatgcgcacggtgcgccgcagaccatcaccgagctgtgcagcgaataccgtaacacccaaatctataccattaacgacaagatcctgagctacaccgagagcatggcgggcaagcgtgaaatggtgatcattaccttcaaaagcggtgaaacctttcaggtggaagttccgggcagccagcacatcgacagccaaaagaaagcgattgagcgtatgaaagataccctgcgtatcacctatctgaccgaaaccaagattgataaactgtgcgtgtggaacaacaagaccccgaacagcatcgcggcgattagcatgaaaaacggtggcagcggtggcgcggcggaggcgagcaagaaaccgcgtcagaagcgtaccgcgaccaaagcgtacaacgttacccaagcgttcggtcgtcgtggtccggagcagacccagggtaactttggcgaccaggaactgatccgtcaaggcaccgattataagcactggccgcagattgcgcagtttgcgccgagcgcgagcgcgttctttggtatgagccgtatcggcatggaagtgaccccgagcggcacctggctgacctacaccggcgcgattaagctggacgataaagacccgaactttaaagatcaagttatcctgctgaacaagcacattgacgcgtataaaacc

SEQ ID NO: 17 - protein, 404 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGGGSNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVD

SEQ ID NO: 18 - protein, 403 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGGGSQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFG

SEQ ID NO: 19 - protein, 697 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGGGSNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGGSGGGGSGGGGSGGGGSQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFG

SEQ ID NO: 20 - protein, 702 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGGGSNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGGSGGGGSGGGGSGGGGSGGGGSQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFG

SEQ ID NO: 21 - protein, 330 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGGGSTNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 22 - protein, 624 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGGGSNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGGSGGGGSGGGGSGGGGSTNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 23 - protein, 629 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGGGSNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGGSGGGGSGGGGSGGGGSGGGGSTNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 24 - protein, 345 aa, Artificial Sequence
  Synthetic
  MISC_FEATURE (334)..(334): X is Cys or Ala
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNEFITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKXVNFNFNGLTGT

SEQ ID NO: 25 - DNA, 402 bp, Artificial Sequence
  Synthetic
atgggcaacaaggtgaaatgctacgttctgttcaccgcgctgctgagcagcctgtatgcgcacggtgcgccgcagaccatcaccgagctgtgcagcgaataccgtaacacccaaatctataccattaacgacaagatcctgagctacaccgagagcatggcgggcaagcgtgaaatggtgatcattaccttcaaaagcggtgaaacctttcaggtggaagttccgggcagccagcacatcgacagccaaaagaaagcgattgagcgtatgaaagataccctgcgtatcacctatctgaccgaaaccaagattgataaactgtgcgtgtggaacaacaagaccccgaacagcatcgcggcgattagcatgaaaaacctcgagcaccaccaccaccaccactga

SEQ ID NO: 26 - protein, 133 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNLEHHHHHH

SEQ ID NO: 27 - DNA, 804 bp, Artificial Sequence
  Synthetic
atgggcaacaaggtgaaatgctacgttctgttcaccgcgctgctgagcagcctgtatgcgcacggtgcgccgcagaccattaccgagctgtgcagcgaataccgtaacacccaaatctataccattaacgacaagatcctgagctacaccgagagcatggcgggcaagcgtgaaatggtgatcattaccttcaaaagcggtgaaacctttcaggtggaagttccgggcagccagcacatcgacagccaaaagaaagcgattgagcgtatgaaagataccctgcgtatcacctatctgaccgaaaccaagattgacaaactgtgcgtgtggaacaacaagaccccgaacagcatcgcggcgattagcatgaaaaacggtggcagcggtggcaacaacaccgcgagctggttcaccgcgctgacccagcacggtaaagaggacctgaaatttccgcgtggtcaaggcgttccgatcaacaccaacagcagcccggacgatcaaattggttactatcgtcgtgcgacccgtcgtatccgtggtggcgacggcaagatgaaggacctgagcccgcgttggtacttctactacctgggcaccggtccggaggcgggtctgccgtatggcgcgaacaaggacggtatcatttgggtggcgaccgaaggcgcgctgaacaccccgaaagatcacattggcacccgtaacccggcgaacaacgcggcgatcgttctgcaactgccgcaaggcaccaccctgccgaagggtttttacgcggaaggcagcctcgagcaccaccaccaccaccac

SEQ ID NO: 28 - protein, 268 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSLEHHHHHH

SEQ ID NO: 29 - DNA, 750 bp, Artificial Sequence
  Synthetic
atgggcaacaaggtgaaatgctacgttctgttcaccgcgctgctgagcagcctgtatgcgcacggtgcgccgcagaccatcaccgagctgtgcagcgaataccgtaacacccaaatctataccattaacgacaagatcctgagctacaccgagagcatggcgggcaagcgtgaaatggtgatcattaccttcaaaagcggtgaaacctttcaggtggaagttccgggcagccagcacatcgacagccaaaagaaagcgattgagcgtatgaaagataccctgcgtatcacctatctgaccgaaaccaagattgataaactgtgcgtgtggaacaacaagaccccgaacagcatcgcggcgattagcatgaaaaacggtggcagcggtggcgcggcggaggcgagcaagaaaccgcgtcagaagcgtaccgcgaccaaagcgtacaacgttacccaagcgttcggtcgtcgtggtccggagcagacccagggtaactttggcgaccaggaactgatccgtcaaggcaccgattataagcactggccgcagattgcgcagtttgcgccgagcgcgagcgcgttctttggtatgagccgtatcggcatggaagtgaccccgagcggcacctggctgacctacaccggcgcgattaagctggacgataaagacccgaactttaaagatcaagttatcctgctgaacaagcacattgacgcgtataaaaccctcgagcaccaccaccaccaccac

SEQ ID NO: 30 - protein, 250 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTLEHHHHHH

(19) United States
(12) Patent Application Publication
(10) Pub. No.: US 2023/0190923 A1
(43) Pub. Date: Jun. 22, 2023

(54) COMPOSITIONS COMPRISING LTB AND PATHOGENIC ANTIGENS, AND USE THEREOF

(71) Applicant: MIGAL GALILEE RESEARCH INSTITUTE LTD., Kiryat Shmona (IL)

(72) Inventors: Jacob PITCOVSKI, Korazim (IL); Ehud SHAHAR, Kfar Giladi (IL); Itai BLOCH, Ramot Naftali (IL); Itamar YADID, Ayelet HaShachar (IL); Ran BEN ADIVA, Ma'ayan Baruch (IL); Chen KATZ, Gonen (IL); Nady GRUZDEV, Kidmat Zvi (IL)

(73) Assignee: MIGAL GALILEE RESEARCH INSTITUTE LTD., Kiryat Shmona (IL)

(21) Appl. No.: 17/802,465

(22) PCT Filed: Feb. 25, 2021
(86) PCT No.: PCT/IL2021/050216

§ 371 Date: Aug. 25, 2022

Related U.S. Application Data
(60) Provisional application No. 62/981,127, filed on Feb. 25, 2020.
(60) Provisional application No. 62/990,196, filed on Mar. 16, 2020.
(60) Provisional application No. 63/109,138, filed on Nov. 3, 2020.

(51) Int. Cl. A61K 39/295; A61P 37/04; C07K 14/005; C07K 14/245; C12N 15/85

(52) U.S. Cl. CPC A61K 39/295; A61P 37/04; C07K 14/005; C07K 14/245; C12N 15/85; A61K 2039/5256

(57) ABSTRACT

The present invention is directed to compositions of immunogenic polypeptides including a heat labile toxin subunit B (LTB) polypeptide and a plurality of viral polypeptides. Further provided are compositions and methods of using same, such as for vaccinating a subject in need thereof.

### CROSS-REFERENCE TO RELATED APPLICATIONS

This application claims the benefit of priority of U.S. Provisional Application No. 62/981,127, titled: “CHIMERIC POLYPEPTIDE COMPRISING LTB AND A PATHOGEN ANTIGEN, COMPOSITIONS COMPRISING SAME, AND USE THEREOF”, filed Feb. 25, 2020, U.S. Provisional Application No. 62/990,196, titled: “CHIMERIC POLYPEPTIDE COMPRISING LTB AND A PATHOGEN ANTIGEN, COMPOSITIONS COMPRISING SAME, AND USE THEREOF”, filed Mar. 16, 2020, and U.S. Provisional Application No. 63/109,138, titled: “COMPOSITIONS COMPRISING LTB AND PATHOGENIC ANTIGENS, AND USE THEREOF”, filed Nov. 3, 2020, the contents of which are incorporated herein by reference in their entirety.

### FIELD OF THE INVENTION

The present invention relates in general to the field of vaccines.

### BACKGROUND

Vaccines are delivered through various administration routes, including parenteral routes like intramuscular or subcutaneous injection, and mucosal routes through intranasal, oral, vaginal, or rectal tract. The benefits of mucosal vaccination include: 1) strong mucosal immunity besides systemic immune responses, which provides the first barrier against those infections initiating at the mucosal surface; 2) better patient compliance due to needle free administration; 3) potential to overcome the barrier of the pre-existing immunity caused by previous parenteral vaccinations.

Among different mucosal routs, oral delivery is advantageous considering its superior patient compliance, easy administration, and mass immunization capacity, especially when it comes to the plant-derived protein antigens and veterinary vaccines.

Live viral vectors are widely used as delivery systems in mucosal vaccination, including adenovirus, attenuated influenza virus, Venezuelan equine virus, and poxvirus vectors. Besides viral vectors, nucleic acid-based vaccines such as plasmid DNA and RNA, are also being developed. The limitations of these vaccination strategies include difficulties in microorganism culturing and some safety concerns such as the possibility of reverting to the virulent state in immunocompromised hosts, as well as potential adverse effects including allergic and autoimmune reactions. Furthermore, the introduction of foreign DNA into the body could affect a cell’s normal protein expression pathways. In contrast, vaccines with protein antigens are intrinsically safer than the whole pathogen-based and DNA-based antigens due to the absence of genetic materials.

Protein antigens are widely exploited in vaccine development to protect against infectious diseases. Currently there have not been any approved oral or intranasal protein vaccines yet, but extensive efforts have been reported on mucosal vaccination with protein-based antigens against various infectious diseases such as influenza, tetanus, diphtheria, hepatitis, HIV, SARS-CoV and MERS-CoV, but most of these protein-based antigens are seriously limited by the generally low stability and immunogenicity to induce concerted humoral and cellular immune responses.

The usage of live bacterial cells as vehicles to deliver recombinant antigens may overcome some of these limitations by activation of the innate immune responses, thus acting as useful immunostimulating adjuvants. Many bacterial species such as attenuated strains of Salmonella enterica, Listeria monocytogenes, Streptococcus gordonii, Vibrio cholerae, Mycobacterium bovis (BCG), Yersinia enterocolitica, Shigella flexnery, as well as different Lactic acid bacteria, have been reported as promising candidates for recombinant protein delivery.

LTB is a non-toxic subunit of LT toxin expressed by enterotoxigenic E. coli strains (ETEC), responsible for binding to the host GM1 ganglioside receptors. Moreover, LTB is known to be strong mucosal adjuvant through T cells activation, although the mechanism by which it acts remains unclear. Both these traits make LTB an attractive candidate for different mucosal vaccination strategies.

LTB was used in several vaccine development studies both as free adjuvant and in fusion to various antigens.

Adjuvant properties of LTB were widely used in the development of anti ETEC vaccines.

LTB was found to be a more potent adjuvant than cholera toxin subunit B (CtxB), stimulating stronger responses to hen egg lysozyme when the two were administered nasally to mice, as assessed by enhanced serum and secretory antibody titers as well as by stimulation of lymphocyte proliferation in spleen and draining lymph nodes.

Similarly, LTB used for intranasal immunization together with herpes simplex virus type 1 (HSV-1) elicited higher serum and mucosal anti-HSV-1 titers comparing with that obtained using CtxB.

Mice, immunized subcutaneously or intradermally with H. pylori urease antigen and separately LTB prior to intragastric challenge with H. pylori, demonstrated high protection against the pathogen and increased levels of specific IgG in serum and IgA in saliva.

The addition of LTB to rKnob of egg drop syndrome adenovirus significantly elevated the antibody response in avian groups (e.g., chicken) vaccinated orally and transcutaneously, however, had no influence in injected groups.

Comparative study of live attenuated Salmonella Enteritidis vaccine candidate secreting LTB (SE-LTB) with a commercial Salmonella Enteritidis (SE) vaccine for efficacy of protection against SE infection in laying hens demonstrated significant reduction in internal egg and internal organ contamination by virulent Salmonella Enteritidis in chickens inoculated orally by SE-LTB compared to chickens inoculated by SE vaccine.

Recombinant LTB significantly increased the immunogenic response against SuHV-1 in mice, especially if used intramuscularly in the concentration of 10 µg/dose compared to SuHV-1 alone.

The adjuvant properties of LTB were also demonstrated in DNA vaccines. H. pylori outer inflammatory protein (oipA) administered intradermally as oipA gene encoded construct together with LTB, promoted a strong Th1 immune response in mice. Regardless of the different immune responses promoted by the various vaccination regimes, all immunized mice had smaller bacterial loads after H. pylori challenge than did the negative controls.

LTB adjuvant properties were used in construction of recombinant viruses.

Immunization with recombinant baculovirus BV-Dual-3M2e-LTB harboring a gene cassette expressing three tandem copies of the highly conserved extracellular domain of influenza M2 protein (M2e) and LTB, resulted in improved survival and decreased lung virus shedding following the exposure to the different influenza H5N1 clades compared with mice inoculated with BV-Dual-3M2e.

Plant-expressed Human Papilloma Virus antigen HPV16L1 and LTB proteins, extracted from transgenic tobacco leaves caused strong mucosal and systemic immune responses in mice following oral immunization. The IgG and IgA levels were higher in mice immunized by both proteins compared to mice immunized only by HPV16L1.

LTB fusions to various antigens expressed in different plants such as Arabidopsis thaliana, carrots, lettuce, rice, and tobacco.

Tobacco plants carrying the LTB:ST (heat stable toxin) gene caused mucosal and systemic humoral responses in mice orally dosed with transgenic tobacco leaves, suggesting plant-derived LTB:ST immunogenicity via the oral route.

Chimeric protein named LTB-EBOV, based on LTB as an immunogenic carrier and two epitopes from the Zaire ebolavirus GP1 protein, expressed in plant tissues induced immunogenic response in BALB/c mice, when administered by either subcutaneous or oral routes. Oral immunization resulted in increased levels of both IgA and IgG antibodies.

LTB fusions were also expressed in bacteria and yeasts. Amino acid sequence from the C-domain of rat synapsin fused to the C-terminal end of LTB, expressed in E. coli and orally delivered to rats was able to elicit a systemic immune response.

A recombinant subunit vaccine (rLTBR1) containing the R repeat region of P97 adhesin of M. hyopneumoniae (R1) fused to LTB expressed in E. coli, produced high levels of systemic and mucosal antibodies in BALB/c mice inoculated by intranasal or intramuscular route. Another study demonstrated that Anti-R1 systemic antibody levels were significantly higher in mice vaccinated with recombinant R1-LTB protein compared to mice vaccinated with R1 alone. Moreover, anti-R1 serum IgA was induced only by rBCG/LTBR1.

Recombinant chimera, consisting of LTB, fused to the C-terminal fragments of botulinum neurotoxins (BoNTs) serotypes C and D, produced in E. coli, induced high levels of neutralizing antibodies against BoNTs in guinea pigs. The same recombinant vaccine also was able to induce strong immunogenic response in cattle.

Administration of the chimeric protein containing three Mycoplasma hyopneumoniae antigens fused to LTB, induced significant IgG and IgA responses against all individual antigens present in the chimaera, but it could not confer a significant protection against M. hyopneumoniae infection in pigs.

BALB/c mice vaccinated in the deep lungs by LTB fusion to mycolyl-transferase antigen 85A, demonstrated promoted a Th-2 biased immune response, with a production of IL-5 but not IFN-γ by spleen mononuclear cells in vitro. Pulmonary vaccination with 85A-LTB tended to decrease bacterial counts in the spleen and lungs following a virulent challenge with M. tuberculosis H37Rv.

Influenza antigen, comprised of a set of consensus influenza A virus epitopes (IAVe), genetically linked to LTB, injected to pigs improved protection against a pathogenic H1N1 swine influenza virus challenge, with about 50% compared to non-immunized animals and caused to extensive humoral and cellular immune responses.

In other study, virus-like particles (VLP) were created by genetic fusion of M2 protein, a conservative transmembrane protein of the avian influenza A virus to the hepatitis B virus core antigen (HBcAg) protein and to LTB. The mice immunization with VPL by intranasal dropping and oral routes revealed that LTB can significantly enhance the mucosal immune responses of mice.

Hamster vaccination with LipL32- a surface lipoprotein present in all pathogenic species of Leptospira coupled to or co-administered with LTB resulted in increased survival following exposure to 5× the 50% lethal dose (LD50) of Leptospira interrogans.

The antigen-specific antibody titer of mice orally administered with toxin epitope produced by Actinobacillus pleuropneumoniae coupled to LTB was increased compared to LTB fusion form or antigen alone administration. Better protection against challenge infection with A. pleuropneumoniae was also observed for coexpression in recombinant yeast compared with others.

In a recent study, mouse model was used to explore the immunogenicity of an oral dengue vaccine candidate prepared using whole recombinant yeast cells (WC) and cell-free extracts (CFE) from cells expressing recombinant LTB fused to the consensus dengue envelope domain III (scEDIII). Although both WC and CFE dosage applications of LTB-scEDIII stimulated a systemic humoral immune response in the form of dengue-specific serum IgG as well as mucosal immune response in the form of secretory sIgA, sera from mice that were fed CFE preparations demonstrated markedly higher anti dengue virus neutralizing titters compared to those from WC-fed mice.

LTB have been also reported as a candidate for the transcutaneous delivery of tumor antigens in cancer immunotherapy. Multiepitope polypeptide (MEP) genetically fused to LTB injected transcutaneously was able to induce anti MEP immunogenic response in mice.

However, even in view of the extensive work invested in the development of novel vaccines, there is still an urgent need for improved vaccines, for example for use in vaccination of subjects afflicted, infected with or at increased risk of infection of a pathogen including but not limited to coronavirus.

### SUMMARY

According to one aspect, the invention provides a composition comprising: a heat labile toxin subunit B (LTB) polypeptide comprising the sequence: MNKVKCYVLFTALLSSLYAHGAPQTITELCSEYRNTQIYTINDKILSYTESMAGKREMVI ITFKSGETFQVEVPGSQHIDSQKKAIERMKDTLRITYLTETKIDKLCVWNNKTPNSIAAIS MKN (SEQ ID NO: 1) or an analog thereof having at least 80% sequence identity to said LTB; and a plurality of immunogenic polypeptides; wherein said plurality of immunogenic polypeptides comprises at least two viral peptides or any analogs thereof having at least 80% sequence identity to said at least two viral peptides.

In some embodiments, the composition comprises a first viral peptide, and said LTB conjugated to at least a second viral peptide, thereby forming a chimeric polypeptide.

In some embodiments, the LTB polypeptide comprises a plurality of LTB polypeptides. In some embodiments, the plurality of LTB polypeptides comprises: (i) at least a first LTB polypeptide being a non-conjugated LTB; (ii) at least a second LTB polypeptide conjugated to at least one peptide of said plurality of immunogenic polypeptides, thereby forming a chimeric polypeptide; or (iii) any combination of (i) and (ii).

In some embodiments, the plurality of LTB polypeptides comprises at least a first LTB polypeptide being a non-conjugated LTB and at least a second LTB polypeptide conjugated to at least one peptide of said plurality of immunogenic polypeptides, thereby forming a chimeric polypeptide.

In some embodiments, the composition comprises at least two viral peptides comprising (a) a viral spike protein; and (b) a viral nucleocapsid protein, wherein said at least two viral peptides comprise the full length amino acid sequence or a partial amino acid sequence of said viral spike protein and of said viral nucleocapsid protein, or an analog of any one of said spike protein and of said nucleocapsid protein, having at least 80% sequence identity to any one of said spike protein and said nucleocapsid protein.

In some embodiments, the spike protein comprises the amino acid sequence of SEQ ID NO: 2; SEQ ID NO: 3; SEQ ID NO: 24; or any analog having at least 80% sequence identity to SEQ ID Nos.: 2, 3, or 24.

In some embodiments, the nucleocapsid protein comprises the amino acid sequence: SEQ ID NO: 4; SEQ ID NO: 5; SEQ ID NO: 6; SEQ ID NO: 7; or any analog having at least 80% sequence identity to SEQ ID NO: 4, SEQ ID NO: 5, SEQ ID NO: 6, or SEQ ID NO: 7.

In some embodiments, the conjugated is via a peptide linker comprising an amino acid sequence of 2 to 10 amino acids. In some embodiments, the linker comprises 3 to 7 amino acids. In some embodiments, the linker comprises Serine and Glycine amino acid residues. In some embodiments, the linker consists of Serine and Glycine amino acid residues.

In some embodiments, the composition comprises: (a) an LTB polypeptide being a non-conjugated LTB; and said at least two viral peptides; (b) a first LTB polypeptide being a non-conjugated LTB; a first viral peptide; and a chimeric polypeptide comprising at least a second LTB polypeptide conjugated to at least a second viral peptide; (c) a first chimeric polypeptide comprising a first LTB polypeptide conjugated to at least a first viral peptide and a second chimeric polypeptide comprising at least a second LTB polypeptide conjugated to at least a second viral peptide; (d) a first viral peptide; and a chimeric polypeptide comprising an LTB polypeptide conjugated to at least a second viral peptide; or any combination of (a) to (d).

In some embodiments, the chimeric polypeptide comprises the sequence of SEQ ID NO: 8. In some embodiments, the chimeric polypeptide comprises the sequence of SEQ ID NO: 9. In some embodiments, the chimeric polypeptide comprises the sequence of SEQ ID NO: 10. In some embodiments, the chimeric polypeptide comprises the sequence of SEQ ID NO: 11. In some embodiments, the chimeric polypeptide comprises the sequence of SEQ ID NO: 12. In some embodiments, the chimeric polypeptide comprises the sequence of SEQ ID NO: 13. In some embodiments, the chimeric polypeptide comprises the sequence of SEQ ID NO: 24.

In some embodiments, the at least two viral peptides comprise at least one immunogenic antigen of a pathogenic virus.

In some embodiments, the pathogenic virus is an animal pathogen. In some embodiments, the animal is selected from the group consisting of: a mammal, an avian, and a fish. In some embodiments, the animal is a human subject.

In some embodiments, the pathogenic virus is a Coronavirus. In some embodiments, the Coronavirus comprises any one of: the Wuhan human Corona 2020 (SARS-CoV-2), SARS-Cov, or MERS-Cov.

In some embodiments, the pathogenic virus is an avian pathogenic virus selected from the group consisting of: Infectious bursal disease virus (IBDV), Infectious bronchitis virus (IBV), Reovirus, Influenza virus, Chicken anemia virus (CAV), Newcastle disease virus (NDV), Marek’s disease virus (MDV), Egg drop syndrome (EDS) avian adenovirus, and hemorrhagic enteritis virus (HEV).

In some embodiments, the at least one immunogenic antigen of IBDV is VP2; of IBV is S1, N, or combination thereof; of Reovirus is Sigma C; of Influenza virus is HA; of CAV is VP1 or VP2; of NDV is HN or F; of EDS is KNOB; and of HEV is KNOB.

According to another aspect, there is provided a pharmaceutical composition comprising the composition of the present invention, and a pharmaceutically acceptable carrier.

In some embodiments, the pharmaceutical composition is for use in for use in vaccination of a subject against a pathogen.

In some embodiments, the pharmaceutical composition is formulated for an administration route selected from the group consisting of: oral, transdermal, anal, nasal, topical, and in-ovo.

In some embodiments, there is provided the pharmaceutical composition of the invention, wherein said subject is infected with or at increased risk of infection of: Coronavirus, SARS-CoV-2, SARS-Cov, MERS-Cov, IBV, IBDV, Reovirus, Influenza virus, CAV, NDV, MDV, EDS avian adenovirus, and HEV.

In some embodiments, there is provided a pharmaceutical composition of the invention, wherein said subject is selected from the group consisting of: a human, an avian, and a fish.

In some embodiments, there is provided a polynucleotide molecule encoding the chimeric polypeptide of the present invention.

In some embodiments, there is provided an expression vector comprising the polynucleotide molecule of the present invention.

In some embodiments, there is provided a cell comprising: (a) the composition of the present invention; (b) the polynucleotide molecule of the present invention; the expression vector of the present invention; or any combination of (a) to (c).

In some embodiments, the cell is a naive cell or a recombinant cell. In some embodiments, the cell further lacking one or more extra-cellular proteases.

In some embodiments, the cell is selected form the group consisting of: a bacterial cell, a plant cell, a mammalian cell, an insect cell, and a yeast cell. In some embodiments, the cell is an E. coli cell.

In some embodiments, the cell further comprises a general secretory pathway (GSP) operon encoding a type II secretion system; and optionally lacking a polynucleotide comprising a gene encoding transcription factor histone-like nucleoid-structuring protein (HNS).

In some embodiments, the cell is an E. coli cell derived from a naive (non-recombinant) E. coli K12 or ER2566.

In some embodiments, there is provided a composition comprising the cell provided herein, and an acceptable carrier.

In some embodiments, there is provided a method for treating or preventing a viral infection in a subject in need thereof, comprising administering to said subject a therapeutically effective amount of: (i) the composition of the invention; or the pharmaceutical composition comprising the chimeric polypeptide of the invention, thereby treating or preventing a viral infection in the subject.

In some embodiments, the administering is by a route selected from the group consisting of: oral, transdermal, anal, nasal, topical, and in-ovo.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a vertical bar graph showing GM1-ELISA for secreted LTB in different E. coli strains. The growth medium of each bacteria was collected after 24 h growth on CAYE medium. Samples were compared to purified LTB standard, and purified bovine serum albumin (BSA) served as control. Filtrate from the wild type E. coli MG1655 strain was used as negative control. Numbers 0-781 indicate concentrations of commercial LTB protein (ng/ml). A, B, and C represent filtrates from MG1655 strains transformed with LTB expression plasmid, where A denotes a wildtype bacterium, B denotes a mutant in the Gsp promoter, and C denotes a HNS mutant that expresses the GSP operon in addition to the LTB protein. The data represent averages and standard deviations from two independent experiments each performed in triplicate.

FIG. 2 includes a micrograph of a Coomassie blue stained-SDS-PAGE showing expression profile of fusion proteins LTB-N. Samples: M - marker, 1-4: expression profile of MG1655 bacteria, 1 - wild strain bacteria containing no expression construct, 2 - bacteria expressing only LTB protein, 3 - bacteria expressing the LTB/N218-326 fusion protein, and 4 -bacteria expressing a protein fusion LTB/N29-160.

FIG. 3 includes a vertical bar graph showing GM1-ELISA for secreted LTB-N fusion proteins. The growth medium of each bacteria was collected after 24 h growth on CAYE medium. Samples were compared to purified LTB standard, and purified BSA served as control. filtrate from the wild type E. coli MG1655 strain was used as negative control. A, B, and C represent filtrates from MG1655 transformed with fusion proteins expression plasmid where A represents LTB / N218-326 fusion, B represents LTB/N29-160 fusion C represents LTB protein only. The data represent averages and standard deviations from two independent experiments each performed in triplicate.

FIG. 4 includes a vertical bar graph depicting kinetics of LTB-N secretion. Numbers 0-1560 indicate reference LTB concentrations (ng/well) NC (negative control) indicate filtrate from wild type MG1655 bacteria. Zero (0) h, 4 h, 24 h and 48 h indicate filtrates from MG1655 strain carrying pGEM/LTB/N218-326 fusions harvested following 0-48 hours, respectively. Data represent average + standard deviation from two independent experiments each performed in triplicate.

FIGS. 5A-5H include micrographs of Coomassie blue stained-SDS-PAGE protein profiling and western blot analysis of chicken serums following injection ad oral immunization. Vaccination groups: (5A) injection of PBS (control); (5B): injection of inactivated IBV virus; (5C): injection of WT LTB protein; (5D): injection of LTB-N 218-326; (5E): Oral intake of WT E. coli MG1655; (5F): Oral intake of E. coli, with its secreted WT LTB; (5G): oral intake of E. coli, with its secreted LTB-N 218-326; and (5H): oral intake of E. coli, with its secreted LTB-N 29-160. The sera of each group were reacted against four samples in a western blot analysis. 1 -Total proteins of WT bacteria; 2 - Partially purified LTB; 3 - Partially purified N 29-160; and 4 -Partially purified N 218-326. Arrows point on the target proteins.

FIG. 6 includes a vertical bar graph showing detection of immunoglobulin Y (IgY) antibodies in chicken serums following injection and oral vaccination. The test was performed by ELISA method using full length purified N protein (right panel). The letters represent pools of serums from each vaccination group (as described in FIG. 5. Presented data from two independent experiments each performed in triplicate).

FIG. 7 includes a vertical bar graph showing fluorescent activated cell sorting (FACS) results calculating percentages of CD8+ cells of total CD3 T cells after incubation of splenocytes from the treated birds with IBV virus (M 41). A, B, C and D represent group of birds vaccinated with control wild type strain, strain expressing LTB alone, strain expressing LTB-N29-160 fusion, and strain expressing LTB- N218-326 fusion, respectively. The data represent the mean percentages of one representative experiment. Bars marked with asterisk indicate significant difference (P<0.05) in percentages between the different groups.

FIG. 8 includes a vertical bar graph showing detection of IgY antibodies in chicken sera following oral vaccination. The test was performed using commercial IBV-ELISA kit according to manufacture instructions. The WT, S, N and N + S symbols represent groups of birds vaccinated with live bacteria carrying appropriate systems. NC and PC represent positive and negative controls, respectively, and Scl represents a group of birds that have been vaccinated with LTB-S1 purified protein. Presented are averages and standard deviations for each group from one representative experiment.

FIG. 9 includes a table showing groups of vaccination and challenge study performed with partially purified chimeric proteins LTB-S1, LTB-N3, and LTB-N5. As described Example 5, hereinbelow.

FIG. 10 includes a graph showing quantitative RT-PCR (qRT-PCR) calibration curve used for the quantification of corona IBV particles by its genetic material. The Y axis represents tested IBV EID50/ml concentrations, and the X axis represents cycle threshold (CT) results in the qRT-PCR reaction.

FIG. 11 includes a vertical bar graph showing antibodies levels in sera of chickens after oral administration of chimeric LTB-IBV antigens. Shown are enzyme-linked immunosorbent assay (ELISA) results for antibody levels against IBV in chicken sera after vaccination and booster in two weeks interval. Chickens were vaccinated (left to right) with inactivated commercial vaccine IBV (M41), protein expression media (NC), E. coli bacteria (WT), LTBN3+LTBN5 proteins, LTBS1 protein, and LTBS1+ LTBN3+ LTBN5 proteins. Results exceeding optical density (O.D)₄₂₀ value of 0.2 (red line) are considered positive for the detection of IBV as stated by manufacturer.

FIGS. 12A-12B include vertical bar graphs showing IBV shedding after challenging chickens vaccinated orally with E. coli secreting chimeric LTB-IBV antigens with M41 virulent IBV. qRT-PCR results derived from trachea and cloaca swabs samples taken at days 0, 3, 6 and 10 post challenge. (12A) Percent of chickens tested positive for shedding M41-IBV. (12B) Virus quantification from swab samples taken from the trachea (top), cloaca (middle) and both cloaca and trachea (bottom). Chickens were vaccinated with protein expression media divided into non-challenged (NC) or challenged (PC), E. coli bacteria (WT), inactivated commercial vaccine (IBV M41), LTBS1, LTBN3+LTBN5 and LTBS1+LTBN3+LTBN5.

FIG. 13 includes a scheme of a non-limiting study design as described herein (see Example 7).

FIG. 14 includes a vertical bar graph showing results of an enzyme-linked immunosorbent assay (ELISA) for the measuring of anti S1 serum IgG titers.

FIG. 15 includes vertical bar graph showing results of ELISA for the measuring of anti S1 faecal IgA levels.

FIG. 16 includes a bar graph showing results of ELISA for the measuring of anti S1 serum IgG titers following a second dose and a third dose of vaccination.

FIG. 17 includes a vertical bar graph showing results of an MMT assay for measuring the proliferation of splenocytes that were induced by a nucleocapsid polypeptide.

FIG. 18 includes a vertical bar graph showing results of an ELISpot assay for measuring the number of specific interferon gamma (INF-γ) secreting T cells after induction by a nucleocapsid polypeptide.

FIG. 19 includes a vertical bar graph showing that oral vaccination with high dose (as described in FIG. 13) results in the production of neutralizing antibodies.

FIGS. 20A-20C depict sequence alignments between the spike protein of SARS-CoV-2 (UniProt Number: PODTC2) and MERS (UniProt Number: K9N5Q8) (20A); SARS-CoV-2 and SARS-CoV-1 (UniProt Number: P59594) (20B); SARS-CoV-2 and IBV (20C).

FIGS. 21A-21E include structure comparison between the spike protein of IBV (21A), MERS (21B), SARS-CoV-1 (21C), and SARS-CoV-2 (21D). (21E) Aligned structure of 21A-21D.

FIGS. 22A-22G include graphs showing the effect of S1 and S1-receptor binding domain (RBD) as immunogenic inducers on rat splenocytes after prior vaccination with Nucleocapsid (negative control), S1-RBD, S1 or LTB proteins. (22A-22D) ELISpot assay for measuring the number of specific interferon gamma (INF-γ) secreting T cells that were induced by a S1 (22A), S1-RBD (22B), and S1 peptide pool (Peptivator™) (22C). (22D) A cumulative comparison of (22A-22C) showing that the greatest activation was observed when using S1 peptide pool. (22E-22F) Graphs showing the titer of serum anti S1 (22E) and anti S 1-RBD (22F) IgGs. (22G) A graph showing serum neutralization.

FIGS. 23A-23D include graphs showing the effect of the nucleocapsid protein (N) as an immunogenic inducer on rat splenocytes after prior vaccination with Nucleocapsid, S1-RBD, S1 or LTB proteins. (23A-23C) ELISpot assay for measuring the number of specific interferon gamma (INF-γ) secreting T cells that were induced by a full N protein (23A), or an N peptide pool (N-Peptivator™) (23B). (23C) A cumulative comparison of (23A-23B). Splenocytes from rats vaccinated with N protein elevated the number of specifically activated T cells after induction with N protein and N peptide pool (Peptivator™). (23D) A graph showing the titer of serum anti N IgG.

FIGS. 24A-24B include graphs showing the effect of LTB as an immunogenic inducer. (24A) ELISpot assay for measuring the number of specific interferon gamma (INF-γ) secreting T cells that were induced by LTB. Splenocytes from rats vaccinated with LTB protein demonstrated relatively high number of specifically activated T cells after induction with LTB protein. (24B) A graph showing the titer of serum anti LTB IgG.

### DETAILED DESCRIPTION

In some embodiments, the present invention is directed to a composition comprising LTB and a plurality of immunogenic polypeptides. In some embodiments, the composition comprises at least one chimeric polypeptide comprising at least a first LTB polypeptide and at least a first viral peptide of the plurality of immunogenic polypeptides.

In some embodiments, there is provided a chimeric polypeptide comprising LTB and at least one immunogenic polypeptide, such as a viral peptide.

In some embodiments, the plurality of immunogenic polypeptides comprise at least one viral peptide derived from a Coronavirus. As used herein, the terms “Coronavirus” or “Coronaviruses” encompass any virus belonging to the family of “Coronaviridae”. In some embodiments, the Coronavirus is selected from any of the genera: Alphacoronavirus, Betacoronavirus, Gammacoronavirus, and Deltacoronavirus.

### Composition

According to some embodiments, there is provided a composition comprising: a heat labile toxin subunit B (LTB) polypeptide and a plurality of immunogenic polypeptides.

In some embodiments, the LTB polypeptide comprises the sequence: MGNKVKCYVLFTALLSSLYAHGAPQTITELCSEYRNTQIYTINDKILSYTESMAGKREM VIITFKSGETFQVEVPGSQHIDSQKKAIERMKDTLRITYLTETKIDKLCVWNNKTPNSIAA ISMKN (SEQ ID NO: 1) or an analog thereof having at least 80% sequence identity to the LTB.

In some embodiments, the composition comprises a first viral peptide, and the LTB polypeptide being conjugated to at least a second viral peptide, thereby forming a chimeric polypeptide.

In some embodiments, the plurality of immunogenic polypeptides comprises at least two viral peptides or any analogs thereof having at least 80% sequence identity to the at least two viral peptides.

According to some embodiments, there is provided a composition comprising LTB or a functional analog thereto (such as, but not limited to CTB) and at least one viral peptide selected from: spike protein 1, nucleocapsid protein, any functional fragment thereof, or any analog thereof having at least 80% homology or identity thereto.

In some embodiments, the spike protein 1 fragment or analog composites the RBD of the spike protein. In some embodiments, the LTB and the at least one viral peptide are non-conjugated to one another.

In some embodiments, the LTB polypeptide comprises a plurality of LTB polypeptides.

In some embodiments, the composition of the invention comprises a plurality of LTB polypeptides.

As used herein, the term “plurality” comprises any integer equal to or greater than 2. In some embodiments, a plurality comprises at least 2, at least 3, at least 5, at least 7, at least 9, at least 10, at least 12, or at least 15, or any value and range therebetween. Each possibility represents a separate embodiment of the invention. In some embodiments, a plurality comprises 2 to 15, 2 to 10, 2 to 8, 2 to 4, 3 to 11, 3 to 9, 3 to 7, 4 to 15, or 4 to 8. Each possibility represents a separate embodiment of the invention.

In some embodiments, the plurality of LTB polypeptides comprises: (i) at least a first LTB polypeptide being a non-conjugated LTB; (ii) at least a second LTB polypeptide conjugated to at least one peptide of the plurality of immunogenic polypeptides, thereby forming a chimeric polypeptide of the invention; or (iii) any combination of (i) and (ii).

As used herein, the term “non-conjugated” refers to a “free” peptide or a polypeptide. LTB. In some embodiments, free peptide, or polypeptide, e.g., LTB refers to a polypeptide, e.g., LTB, that is not fused to any other peptide as described herein. In some embodiments, a free LTB is devoid of any portion or partial sequence of a viral peptide as described herein, conjugated of fused thereto. In some embodiments, a free LTB comprises any LTB, such as a wildtype LTB, a modified LTB, a tag comprising LTB (such as for purification or identification), as long as the free LTB is devoid of any portion or partial sequence of a viral peptide as described herein.

In some embodiments, any of the proteins and/or polypeptides disclosed herein may comprise a tag or a flag such as for purification and/or isolation and/or identification.

In some embodiments, the tag is located at the N terminal end, the C terminal end, or on both ends of any one of the proteins and/or polypeptides disclosed herein.

In some embodiments, the tag is a Histidine tag (His tag). In some embodiments, a His tag comprises 6 to 10 histidine residues. In some embodiments, a His tag comprises 6 to 8 histidine residues.

As used herein, the term “conjugated” refers to a peptide or a polypeptide, e.g., LTB, being linked to at least one different peptide, e.g., the plurality of immunogenic peptides. In some embodiments, linked comprises chemically bound. In some embodiments, a chemical bond comprises a covalent bond. In some embodiments, a chemical bond comprises a peptide bond. In some embodiments, LTB is linked to the at least one peptide of the plurality of immunogenic peptides directly. In some embodiments, LTB is linked to the at least one peptide of the plurality of immunogenic peptides indirectly, such as via a linker, as disclosed herein. In some embodiments, the linker is a flexible or a rigid linker.

In some embodiments, the plurality of LTB polypeptides comprises at least a first LTB polypeptide being a non-conjugated LTB and at least a second LTB polypeptide conjugated to at least one peptide of the plurality of immunogenic polypeptides, thereby forming a chimeric polypeptide of the invention.

In some embodiments, the immunogenic polypeptides of the plurality of immunogenic polypeptides are derived from a single pathogen species. In some embodiments, the immunogenic polypeptides of the plurality of immunogenic polypeptides are derived from multiple pathogen species.

In some embodiments, the at least two viral peptides comprise (a) a viral spike protein; and (b) a viral nucleocapsid protein.

In some embodiments, the at least two viral peptides are derived from a single virus species. In some embodiments, the at least two viral peptides are derived from multiple virus species.

In some embodiments, the spike protein and the nucleocapsid protein are derived from a single virus species. In some embodiments, the spike protein and the nucleocapsid protein are derived from multiple virus species. In some embodiments, the spike protein is derived from a first virus species and the nucleocapsid protein is derived from a second virus species.

In some embodiments, the at least two viral peptides comprise the full-length amino acid sequence or a partial amino acid sequence of the viral spike protein and of the viral nucleocapsid protein.

In some embodiments, the at least two viral peptides comprise an analog of any one of: the spike protein, and the nucleocapsid protein.

In some embodiments, the spike protein comprises any viral spike protein. In some embodiments, the spike protein comprises any coronavirus derived spike protein. In some embodiments, the spike protein comprises any coronavirus derived spike protein as long as the spike protein is essentially structurally similar or identical to the SARS-CoV-2 spike protein.

In some embodiments, essentially structurally identical is determined according to the level of the root mean square distancing (RMSD). In some embodiments, any viral spike protein being essentially structurally similar or identical to the SARS-CoV-2 spike protein is contemplated according to the herein disclosed composition, and methods of using same.

In some embodiments, the spike protein has a RMSD of 0.1 at most, 0.2 at most, 0.3 at most, 0.4 at most, 0.5 at most, 0.7 at most, 0.9 at most, 1.0 at most, 1.2 at most, 1.4 at most, 1.5 at most, 1.6 at most, 1.8 at most, 1.9 at most, 2.0 at most, 2.2 at most, 2.5 at most, or 3.5 at most, corresponding to the SARS-CoV-2 spike protein, or any value and range therebetween. Each possibility represents a separate embodiment of the invention.

In some embodiments, the analog has at least 15%, at least 20%, at least 30%, at least 40%, at least 50%, at least 60%, at least 70%, at least 80%, or at least 90% sequence identity to any one of: the spike protein and the nucleocapsid protein, or any value and range therebetween. Each possibility represents a separate embodiment of the invention.

In some embodiments, the composition of the invention comprises: a first LTB polypeptide being a non-conjugated LTB; and the at least two viral peptides. In some embodiments, the composition comprises a first LTB polypeptide being a non-conjugated LTB and the at least two viral peptides unconjugated to the first LTB polypeptide.

In some embodiments, the composition comprises a first LTB polypeptide being a non-conjugated LTB; a first viral peptide; and a chimeric polypeptide comprising at least a second LTB polypeptide conjugated to at least a second viral peptide, as disclosed herein.

In some embodiments, the composition comprises a first chimeric polypeptide comprising a first LTB polypeptide conjugated to at least a first viral peptide and a second chimeric polypeptide comprising at least a second LTB polypeptide conjugated to at least a second viral peptide, as disclosed herein.

In some embodiments, the composition comprises any combination of: a first LTB polypeptide being a non-conjugated LTB; and the at least two viral peptides, a first LTB polypeptide being a non-conjugated LTB; a first viral peptide; and a chimeric polypeptide comprising at least a second LTB polypeptide conjugated to at least a second viral peptide, and a first chimeric polypeptide comprising a first LTB polypeptide conjugated to at least a first viral peptide and a second chimeric polypeptide comprising at least a second LTB polypeptide conjugated to at least a second viral peptide.

In some embodiments, the composition comprises a first LTB polypeptide being a non-conjugated LTB, a first viral peptide, being a non-conjugated or free spike protein, as described herein, and a second viral peptide, being a non-conjugated or free nucleocapsid protein, as described herein.

In some embodiments, the composition comprises a first LTB polypeptide being a non-conjugated LTB; a first viral peptide, a non-conjugated or free spike protein, as described herein; and a chimeric polypeptide comprising at least a second LTB polypeptide conjugated to at least a second viral peptide, being a nucleocapsid protein, as described herein.

In some embodiments, the composition comprises a first chimeric polypeptide comprising a first LTB polypeptide conjugated to at least a first viral peptide, being a spike protein, as described herein, and a second chimeric polypeptide comprising at least a second LTB polypeptide conjugated to at least a second viral peptide, being a nucleocapsid protein, as described herein.

In some embodiments, the at least two viral peptides comprise at least one immunogenic antigen of a pathogenic virus. In some embodiments, the at least two viral peptides comprise at least 2, at least 3, at least 4, at least 5, at least 6, at least 7, at least 8, at least 9, or at least 10 immunogenic antigens, or any value and range therebetween. Each possibility represents a separate embodiment of the invention.

In some embodiments, the composition further comprises a CTB polypeptide, as described herein. In some embodiments, the composition further comprises a CTB polypeptide. In some embodiments, the composition comprises an LTB polypeptide or a CTB polypeptide. In some embodiments, the composition of the invention comprises a plurality of immunogenic peptides with LTB or CTB. In some embodiments, LTB and CTB are alternatives for use in the herein disclosed composition. In some embodiments, a composition comprising LTB is devoid of CTB. In some embodiments, a composition comprising CTB is devoid of LTB. In some embodiments, the composition is formulated with LTB or CTB as equivalent alternatives.

In some embodiments, there is provided a pharmaceutical composition comprising the composition of the invention and a pharmaceutically acceptable carrier.

In some embodiments, there is provided a pharmaceutical composition comprising: (a) the chimeric polypeptide of the invention; or (b) the herein disclosed cell, and a pharmaceutically acceptable carrier.

In some embodiments, the pharmaceutical composition comprises (a) a chimeric polypeptide comprising the LTB or CTB polypeptide and a viral spike peptide, (b) a chimeric polypeptide comprising the LTB or CTB polypeptide and a viral nucleocapsid protein, (c) a chimeric polypeptide comprising the LTB or CTB polypeptide, a viral spike peptide, and a viral nucleocapsid protein, or any combination of (a) to (c), and a pharmaceutically acceptable carrier.

In some embodiments, the pharmaceutical composition comprises a cell or a plurality of cells, comprising: (a) a chimeric polypeptide comprising the LTB or CTB polypeptide and a viral spike peptide, (b) a chimeric polypeptide comprising the LTB or CTB polypeptide and a viral nucleocapsid protein, (c) a chimeric polypeptide comprising the LTB or CTB polypeptide, a viral spike peptide, and a viral nucleocapsid protein, or any combination of (a) to (c), and a pharmaceutically acceptable carrier.

In some embodiments, the pharmaceutical composition is for use in the vaccination of a subject against a pathogen. In some embodiments, the pharmaceutical composition is for use in the treatment or prevention of a viral infection in a subject in need thereof.

In some embodiments, the pharmaceutical composition is formulated for an administration route selected from: oral, transdermal, anal, nasal, topical, and in-ovo.

The term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable, or synthetic origin, such as peanut oil, soybean oil, mineral oil, sesame oil and the like, polyethylene glycols, glycerine, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The composition, if desired, can also contain minor amounts of wetting or emulsifying agents, or pH buffering agents such as acetates, citrates, or phosphates. Antibacterial agents such as benzyl alcohol or methyl parabens; antioxidants such as ascorbic acid or sodium bisulfide; and agents for the adjustment of tonicity such as sodium chloride or dextrose are also envisioned.

The compositions can take the form of solutions, suspensions, emulsions, tablets, pills, capsules, powders, gels, creams, ointments, foams, pastes, sustained-release formulations, and the like. The compositions can be formulated as a suppository, with traditional binders and carriers such as triglycerides, microcrystalline cellulose, gum tragacanth or gelatine. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington’s Pharmaceutical Sciences″ by E. W. Martin, the contents of which are hereby incorporated by reference herein. Such compositions will contain a therapeutically effective amount of the peptide of the invention, preferably in a substantially purified form, together with a suitable amount of carrier so as to provide the form for proper administration to the subject.

An embodiment of the invention relates to a polypeptide presented in unit dosage form and are prepared by any of the methods well known in the art of pharmacy. In an embodiment of the invention, the unit dosage form is in the form of a tablet, capsule, lozenge, wafer, patch, ampoule, vial, or pre-filled syringe. In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient’s circumstances. Effective doses can be extrapolated from dose-response curves derived from in-vitro or in-vivo animal model test bioassays or systems.

In an embodiment of the invention, polypeptides are administered via oral, rectal, vaginal, topical, nasal, ophthalmic, transdermal, subcutaneous, intramuscular, intraperitoneal, or intravenous routes of administration. The route of administration of the pharmaceutical composition will depend on the disease or condition to be treated. Suitable routes of administration include, but are not limited to, oral, or parenteral injections, e.g., intradermal, intravenous, intramuscular, intralesional, subcutaneous, intrathecal, and any other mode of injection as known in the art. Although the bioavailability of peptides administered by other routes can be lower than when administered via parenteral injection, by using appropriate formulations it is envisaged that it will be possible to administer the compositions of the invention via transdermal, oral, rectal, vaginal, topical, nasal, inhalation, and ocular modes of treatment. In addition, it may be desirable to introduce the pharmaceutical compositions of the invention by any suitable route, including oral, intraventricular, and intrathecal injection; intraventricular injection may be facilitated by an intraventricular catheter, for example, attached to a reservoir.

For oral applications, the pharmaceutical composition may be in the form of drops, tablets, or capsules, which can contain any of the following ingredients, or compounds of a similar nature: a binder such as microcrystalline cellulose, gum tragacanth or gelatine; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. The tablets of the invention can further be film coated.

The compositions of the present invention are generally administered in the form of a pharmaceutical composition comprising at least one of the active components of this invention, e.g., the chimeric polypeptide, a cell comprising the chimeric polypeptide of the invention, together with a pharmaceutically acceptable carrier or diluent. Thus, the compositions of this invention can be administered either individually or together in any conventional oral, parenteral, or transdermal dosage form.

In some embodiments, the subject is infected with or at increased risk of infection of: Coronavirus, SARS-CoV, SARS-CoV-2, MERS-CoV, IBV, IBDV, Reovirus, Influenza virus, CAV, NDV, MDV, EDS avian adenovirus, HEV, or other human pathogens, e.g., Hepatitis B surface antigen (HBsAg), and others.

In some embodiments, the subject is selected from: a human, an avian, and a fish.

### Chimeric Polypeptides

In some embodiments, the first polypeptide comprises a heat labile toxin subunit B (LTB) polypeptide comprising the sequence:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKN (SEQ ID NO: 1)

, or an analog thereof having at least 80% sequence identity to the LTB, as long as the analog has the activity of the LTB, e.g., insertion of the second polypeptide across a cell membrane.

In some embodiments, the LTB polypeptide comprises the amino acid sequence:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNLEHHHHHH (SEQ ID NO: 26).

In some embodiments, there is provided a polynucleotide encoding the LTB. In some embodiments, the polynucleotide comprises the nucleic acid sequence:

ATGGGCAACAAGGTGAAATGCTACGTTCTGTTCACCGCGCTGCTGAGCAG
CCTGTATGCGCACGGTGCGCCGCAGACCATTACCGAGCTGTGCAGCGAAT
ACCGTAACACCCAAATCTATACCATTAACGACAAGATCCTGAGCTACACC
GAGAGCATGGCGGGCAAGCGTGAAATGGTGATCATTACCTTCAAAAGCGG
TGAAACCTTTCAGGTGGAAGTTCCGGGCAGCCAGCACATCGACAGCCAAA
AGAAAGCGATTGAGCGTATGAAAGATACCCTGCGTATCACCTATCTGACC
GAAACCAAGATTGACAAACTGTGCGTGTGGAACAACAAGACCCCGAACAG
CATCGCGGCGATTAGCATGAAAAAC (SEQ ID NO: 14).

In some embodiments, the polynucleotide comprises the nucleic acid sequence:

ATGGGCAACAAGGTGAAATGCTACGTTCTGTTCACCGCGCTGCTGAGCAG
CCTGTATGCGCACGGTGCGCCGCAGACCATCACCGAGCTGTGCAGCGAAT
ACCGTAACACCCAAATCTATACCATTAACGACAAGATCCTGAGCTACACC
GAGAGCATGGCGGGCAAGCGTGAAATGGTGATCATTACCTTCAAAAGCGG
TGAAACCTTTCAGGTGGAAGTTCCGGGCAGCCAGCACATCGACAGCCAAA
AGAAAGCGATTGAGCGTATGAAAGATACCCTGCGTATCACCTATCTGACC
GAAACCAAGATTGATAAACTGTGCGTGTGGAACAACAAGACCCCGAACAG
CATCGCGGCGATTAGCATGAAAAACCTCGAGCACCACCACCACCACCACT
GA (SEQ ID NO: 25).

In some embodiments, the first polypeptide comprises a Cholera toxin B subunit (CTB), or an analog thereof having at least 80% sequence identity to the CTB, as long as the analog has the activity of the CTB, e.g., insertion of the second polypeptide across a cell membrane.

In some embodiments, the first polypeptide and the second polypeptide are operably linked. In some embodiments, the first polypeptide and the second polypeptide are continuously linked, or contiguous. In some embodiments, the first polypeptide and the second polypeptide are linked via a linker. In some embodiments, the linker is a polypeptide linker. In some embodiments, a polypeptide linker is a dipeptide or longer.

In some embodiments, the second polypeptide comprises at least one a viral peptide. In some embodiments, a viral peptide is any peptide produced, secreted, or derived from a virus. In some embodiments, a viral peptide is a synthetic (e.g., recombinant) peptide substantially identical to a peptide naturally occurring within a virus.

In some embodiments, the viral peptide is a viral fusion peptide, wherein the fusion peptide comprises a plurality of viral derived peptides. In some embodiments, a plurality comprises at least 2, at least 3, at least 5, at least 7, at least 8, or at least 10, or any value and range there between. Each possibility represents a separate embodiment of the invention.

In one embodiment, the viral fusion peptide comprises two viral polypeptides. In some embodiments, a viral fusion peptide comprises a spike protein fused to a nucleocapsid protein.

As used herein, the term “fused” refers to a case wherein two distinct polypeptides are a single continuous chain of amino acids, e.g., a polypeptide comprising the amino acid sequence of both, one after the other.

In some embodiments, the fusion polypeptide is encoded by a single chimeric polynucleotide comprising the coding region of each viral gene operably linked to one another. In some embodiments, the fusion polypeptide is produced by expressing the aforementioned chimeric polynucleotide in a compatible expression system as discussed hereinbelow. In some embodiments, the fusion polypeptide is produced by ligating each of the distinct viral peptides so as to obtain a single fused polypeptide. In some embodiments, a linker is located between the distinct viral peptides.

In some embodiments, the viral peptide comprises a partial sequence or portion of a viral peptide, as long as the partial sequence or portion of the peptide comprises or consists of a defined or an intact structural motif or domain.

In some embodiments, the partial sequence or portion of the peptide is able to keep the polypeptide in a stable and/or soluble conformation. In some embodiments, the present invention is further directed to the selection of a viral peptide or a partial sequence thereof, as long as viral peptide or the partial sequence thereof maintain a stable and/or soluble conformation in vitro. In some embodiments, the present invention is further directed to the selection of a viral peptide or a partial sequence thereof, as long as viral peptide or the partial sequence thereof maintains a stable and/or soluble conformation in vivo. In some embodiments, the present invention is further directed to the selection of a viral peptide or a partial sequence thereof, as long as viral peptide or the partial sequence thereof maintain a stable soluble conformation induce immunogenic response of a host. In some embodiments, the present invention is further directed to the selection of a viral peptide or a partial sequence thereof, as long as viral peptide or the partial sequence thereof maintain a stable soluble conformation a subject administered with the viral peptide or the partial sequence thereof. In some embodiments, wherein a partial viral peptide sequence is selected as the second polypeptide of the chimeric polypeptide of the invention, the partial sequence is selected based on its structural similarity or homology to the structure of the full viral peptide. In some embodiments, the partial viral sequence peptide is a complete domain of the full viral peptide. In some embodiments, the partial viral sequence peptide has a structure or natively folds substantially similar the corresponding amino acids within the full viral peptide. The suitability of a partial viral sequence peptide to be used according to the herein disclosed methods, can be determined based on anyone of stability (e.g., biological half-life), predicted or determined structural similarity (e.g., bioinformatics based on resolved structures, x-ray diffraction, etc.), and solubility.

In some embodiments, a viral peptide is a viral spike protein, a viral nucleocapsid protein, a fusion peptide of both, or any fragment or domain thereof. In some embodiments, a viral peptide comprises the full-length amino acid sequence or a partial amino acid sequence of the viral peptide. In some embodiments, a viral peptide in an analog of a viral peptide having at least 80% sequence identity as long as the analog has the activity of the viral peptide, e.g., immunogenic antigen inducing IgG production.

As used herein, the term fragment refers to any amino acid sequence comprising 10 to 100, 10 to 200, 10 to 300, 10 to 400, 10 to 500, 10 to 600, or 10 to 650 amino acids derived from a viral spike protein or a viral nucleocapsid protein.

In some embodiments, the viral peptide is a domain derived from a viral spike protein or a viral nucleocapsid protein.

In some embodiments, the viral peptide is or comprises the receptor binding domain (RBD) of a viral spike protein.

In some embodiments, the spike protein comprises the amino acid sequence:

VNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRD
LPQGFSALEPLVDLPIGINITRFQTLLALHRSYLTPGDSSSGWTAGAAAY
YVGYLQPRTFLLKYNENGTITDAVDCALDPLSETKCTLKSFTVEKGIYQT
SNFRVQPTESIVRFPNITNLCPFGEVFNATRFASVYAWNRKRISNCVADY
SVLYNSASFSTFKCYGVSPTKLNDLCFTNVYADSFVIRGDEVRQIAPGQT
GKIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLSF
ELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIP
IGAGICASYQTQTNSPRRAR (SEQ ID NO: 2).

In some embodiments, the spike protein comprises the amino acid sequence:

ALYDSSSYVYYYQSAFRPPDGWHLHGGAYAVVNISSESNNAGSSSGCTVG
IIHGGRVVNASSIAMTAPSSGMAWSSSQFCTAYCNFSDTTVFVTHCYKHG
GCPITGMLQQHSIRVSAMKNGQLFYNLTVSVAKYPTFKSFQCVNNLTSVY
LNGDLVYTSNETTDVTSAGVYFKAGGPITYKVMREVRALAYFVNGTAQDV
ILCDGSPRGLLACQYNTGNFSDGFYPFTNSSLVKQKFIVYRENSVNTTFT
LHNFTFHNETGANPNPSGVQNIQTYQTQTAQSGYYNFNFSFLSSFVYKES
NFMYGSYHPSCNFRLETINNGLWFNSLSVSIAYGPLQGGCKQSVFSGRAT
CCYAYSYGGPLLCKGVYSGELDHNFECGLLVYVTKSGGSRIQTATEPPVI
TQHNYNNITLNTCVDYNIYGRTGQGFITNVTDSAVSYNYLADAGLAILDT
SGSIDIFVVQSEYGLNYYKVNPCEDVNQQFVVSGGKLVGILTSRNETGSQ
LLENQFYIKITNGTRRFRR (SEQ ID NO: 3).

In some embodiments, the spike protein comprises the amino acid sequence:

EFITNLCPFGEVFNATRFASVYAWNRKRISNCVADYSVLYNSASFSTFKC
YGVSPTKLNDLCFTNVYADSFVIRGDEVRQIAPGQTGKIADYNYKLPDDF
TGCVIAWNSNNLDSKVGGNYNYLYRLFRKSNLKPFERDISTEIYQAGSTP
CNGVEGFNCYFPLQSYGFQPTNGVGYQPYRVVVLSFELLHAPATVCGPKK
STNLVKNKXVNFNFNGLTGT (SEQ ID NO: 24)

wherein X is cysteine or alanine.

In some embodiments, the spike protein RBD comprises the amino acid sequence set forth in SEQ ID NO: 24.

In some embodiments, the spike protein comprises any analog having at least 80% sequence identity to SEQ ID NO: 2 or SEQ ID NO: 3.

In some embodiments, the spike protein comprises any analog having at least 80% sequence identity to SARS-CoV Spike protein (e.g., UniProt Number: P59594).

In some embodiments, the spike protein comprises any analog having at least 80% sequence identity to MERS-CoV Spike protein (e.g., UniProt Number: K9N5Q8).

In some embodiments, the nucleocapsid protein comprises the amino acid sequence:

NNTASWFTALTQHGKEDLKFPRGQGVPINTNSSPDDQIGYYRRATRRIRG
GDGKMKDLSPRWYFYYLGTGPEAGLPYGANKDGIIWVATEGALNTPKDHI
GTRNPANNAAIVLQLPQGTTLPKGFYAEGS (SEQ ID NO: 4).

In some embodiments, the nucleocapsid protein comprises the amino acid sequence:

AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKT (SEQ ID NO: 5).

In some embodiments, the nucleocapsid protein comprises the amino acid sequence:

VGSSGNASWFQALKAKKLNSPPPKFEGSGVPDNENLKLSQQHGYWRRQAR
YKPGKGGKKSVPDAWYFYYTGTGPAADLNWGDSQDGIVWVSAKGADTKSR
SNQGTRDPDKFDQYPLRFSDGGPDGNFRWDFIPI (SEQ ID NO: 6).

In some embodiments, the nucleocapsid protein comprises the amino acid sequence:

KADEMAHRRYCKRTIPPGYKVDQVFGPRTKGKEGNFGDDKMNEEGIKDGR
VIAMLNLVPSSHACLFGSRVTPKLQPDGLHLRFEFTTVVSRDDPQFDNYV
KICDQCVDG (SEQ ID NO: 7).

In some embodiments, the nucleocapsid protein comprises the amino acid sequence:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGSGGNNTASWFTALTQHGKEDLKF
PRGQGVPINTNSSPDDQIGYYRRATRRIRGGDGKMKDLSPRWYFYYLGTG
PEAGLPYGANKDGIIWVATEGALNTPKDHIGTRNPANNAAIVLQLPQGTT
LPKGFYAEGSLEHHHHHH (SEQ ID NO: 28).

In some embodiments, the nucleocapsid protein comprises the amino acid sequence:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGSGGAAEASKKPRQKRTATKAYNV
TQAFGRRGPEQTQGNFGDQELIRQGTDYKHWPQIAQFAPSASAFFGMSRI
GMEVTPSGTWLTYTGAIKLDDKDPNFKDQVILLNKHIDAYKTLEHHHHHH
(SEQ ID NO: 30).

In some embodiments, the nucleocapsid protein comprises any analog having at least 80% sequence identity to SEQ ID NO: 4, SEQ ID NO: 5, SEQ ID NO: 6, SEQ ID NO: 7, SEQ ID NO: 28, or SEQ ID NO: 30.

In some embodiments, the linker comprises an amino acid sequence of 2 to 10 amino acids. In some embodiments, the linker comprises 3 to 7 amino acids. In some embodiments, the linker comprises or consists of Serine and Glycine amino acid residues.

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGSGGQCVNLTTRTQLPPAYTNSFT
RGVYYPDKVFRSSVLHSTQDLFLPFFSNVTWFHAIHVSGTNGTKRFDNPV
LPFNDGVYFASTEKSNIIRGWIFGTTLDSKTQSLLIVNNATNVVIKVCEF
QFCNDPFLGVYYHKNNKSWMESEFRVYSSANNCTFEYVSQPFLMDLEGKQ
GNFKNLREFVFKNIDGYFKIYSKHTPINLVRDLPQGFSALEPLVDLPIGI
NITRFQTLLALHRSYLTPGDSSSGWTAGAAAYYVGYLQPRTFLLKYNENG
TITDAVDCALDPLSETKCTLKSFTVEKGIYQTSNFRVQPTESIVRFPNIT
NLCPFGEVFNATRFASVYAWNRKRISNCVADYSVLYNSASFSTFKCYGVS
PTKLNDLCFTNVYADSFVIRGDEVRQIAPGQTGKIADYNYKLPDDFTGCV
IAWNSNNLDSKVGGNYNYLYRLFRKSNLKPFERDISTEIYQAGSTPCNGV
EGFNCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLE
ILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRR
AR (SEQ ID NO: 8).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGSGGNNTASWFTALTQHGKEDLKF
PRGQGVPINTNSSPDDQIGYYRRATRRIRGGDGKMKDLSPRWYFYYLGTG
PEAGLPYGANKDGIIWVATEGALNTPKDHIGTRNPANNAAIVLQLPQGTT
LPKGFYAEGS (SEQ ID NO: 9).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGSGGAAEASKKPRQKRTATKAYNV
TQAFGRRGPEQTQGNFGDQELIRQGTDYKHWPQIAQFAPSASAFFGMSRI
GMEVTPSGTWLTYTGAIKLDDKDPNFKDQVILLNKHIDAYKT
(SEQ ID NO: 10).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGSGGALYDSSSYVYYYQSAFRPPD
GWHLHGGAYAVVNISSESNNAGSSSGCTVGIIHGGRVVNASSIAMTAPSS
GMAWSSSQFCTAYCNFSDTTVFVTHCYKHGGCPITGMLQQHSIRVSAMKN
GQLFYNLTVSVAKYPTFKSFQCVNNLTSVYLNGDLVYTSNETTDVTSAGV
YFKAGGPITYKVMREVRALAYFVNGTAQDVILCDGSPRGLLACQYNTGNF
SDGFYPFTNSSLVKQKFIVYRENSVNTTFTLHNFTFHNETGANPNPSGVQ
NIQTYQTQTAQSGYYNFNFSFLSSFVYKESNFMYGSYHPSCNFRLETINN
GLWFNSLSVSIAYGPLQGGCKQSVFSGRATCCYAYSYGGPLLCKGVYSGE
LDHNFECGLLVYVTKSGGSRIQTATEPPVITQHNYNNITLNTCVDYNIYG
RTGQGFITNVTDSAVSYNYLADAGLAILDTSGSIDIFVVQSEYGLNYYKV
NPCEDVNQQFVVSGGKLVGILTSRNETGSQLLENQFYIKITNGTRRFRR
(SEQ ID NO: 11).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGSGGVGSSGNASWFQALKAKKLNS
PPPKFEGSGVPDNENLKLSQQHGYWRRQARYKPGKGGKKSVPDAWYFYYT
GTGPAADLNWGDSQDGIVWVSAKGADTKSRSNQGTRDPDKFDQYPLRFSD
GGPDGNFRWDFIPI (SEQ ID NO: 12).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGSGGKADEMAHRRYCKRTIPPGYK
VDQVFGPRTKGKEGNFGDDKMNEEGIKDGRVIAMLNLVPSSHACLFGSRV
TPKLQPDGLHLRFEFTTVVSRDDPQFDNYVKICDQCVDG
(SEQ ID NO: 13).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGGGSNLTTRTQLPPAYTNSFTRGV
YYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINIT
RFQTLLALHRSYLTPGDSSSGWTAGAAAYYVGYLQPRTFLLKYNENGTIT
DAVD (SEQ ID NO: 17).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGGGSQPTESIVRFPNITNLCPFGE
VFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPC
SFG (SEQ ID NO: 18).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGGGSNLTTRTQLPPAYTNSFTRGV
YYPDKVFRSSVLHSTQDLFLPFF SNVTWFHAIHVSGTNGTKRFDNPVLP
FNDGVYFASTEKSNIIRGWIFGTTLDSKTQSLLIVNNATNVVIKVCEFQF
CNDPFLGVYYHKNNKSWMESEFRVYSSANNCTFEYVSQPFLMDLEGKQGN
FKNLREFVFKNIDGYFKIYSKHTPINLVRDLPQGFSALEPLVDLPIGINI
TRFQTLLALHRSYLTPGDSSSGWTAGAAAYYVGYLQPRTFLLKYNENGTI
TDAVDGGGGSGGGGSGGGGSGGGGSQPTESIVRFPNITNLCPFGEVFNAT
RFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKV
GGNYNYLYRLFRKSNLKPFERDISTEIYQAGSTPCNGVEGFNCYFPLQSY
GFQPTNGVGYQPYRVVVLSFELLHAPATVCGPKKSTNLVKNKCVNFNFNG
LTGTGVLTESNKKFLPFQQFGRDIADTTDAVRDPQTLEILDITPCSFG
(SEQ ID NO: 19).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGGGSNLTTRTQLPPAYTNSFTRGV
YYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNF
KNLREFVFKNIDGYFKIYSKHTPINLVRDLPQGF SALEPLVDLPIGINI
TRFQTLLALHRSYLTPGDSSSGWTAGAAAYYVGYLQPRTFLLKYNENGTI
TDAVDGGGGSGGGGSGGGGSGGGGSGGGGSQPTESIVRFPNITNLCPFGE
VFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPC
SFG (SEQ ID NO:20).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGGGSTNLCPFGEVFNATRFASVYA
WNRKRISNCVADYSVLYNSASFSTFKCYGVSPTKLNDLCFTNVYADSFVI
RGDEVRQIAPGQTGKIADYNYKLPDDFTGCVIAWNSNNLDSKVGGNYNYL
YRLFRKSNLKPFERDISTEIYQAGSTPCNGVEGFNCYFPLQSYGFQPTNG
VGYQPYRVVVLSFELLHAPATVCGPKKSTN (SEQ ID NO: 21).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGGGSNLTTRTQLPPAYTNSFTRGV
YYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINIT
RFQTLLALHRSYLTPGDSSSGWTAGAAAYYVGYLQPRTFLLKYNENGTIT
DAVDGGGGSGGGGSGGGGSGGGGSTNLCPFGEVFNATRFASVYAWNRKRI
SNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRK
SNLKPFERDISTEIYQAGSTPCNGVEGFNCYFPLQSYGFQPTNGVGYQPY
RVVVLSFELLHAPATVCGPKKSTN (SEQ ID NO: 22).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGGGSNLTTRTQLPPAYTNSFTRGV
YYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINIT
RFQTLLALHRSYLTPGDSSSGWTAGAAAYYVGYLQPRTFLLKYNENGTIT
DAVDGGGGSGGGGSGGGGSGGGGSGGGGSTNLCPFGEVFNATRFASVYAW
NRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLY
RLFRKSNLKPFERDISTEIYQAGSTPCNGVEGFNCYFPLQSYGFQPTNGV
GYQPYRVVVLSFELLHAPATVCGPKKSTN (SEQ ID NO: 23).

In some embodiments, the chimeric polypeptide comprises the sequence of:

MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNEFITNLCPFGEVFNATRFASVYAWN
RKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYR
LFRKSNLKPFERDISTEIYQAGSTPCNGVEGFNCYFPLQSYGFQPTNGVG
YQPYRVVVLSFELLHAPATVCGPKKSTNLVKNKXVNFNFNGLTGT (SEQ
ID NO: 24),

wherein X is cysteine or alanine.

In some embodiments, the second polypeptide comprises an immunogenic antigen of a pathogenic virus, bacterium, or a fungus.

As used herein, the term “immunogenic” refers to any compound inducing or priming the immune system of a host subject to produce immunoglobulins targeting the immunogenic antigen.

In some embodiments, the pathogen is an animal pathogen. In some embodiments, the pathogen is not a plant pathogen. In some embodiments, the animal is selected from: a mammal, an avian, and a fish. In some embodiments, the mammal is a human subject. In some embodiments, the animal pathogen virus is or comprises a Coronavirus. In some embodiments, the Coronavirus is or comprises: the Wuhan human Corona 2020 (SARS-CoV-2), SARS-CoV, or MERS-CoV. In some embodiments, the Coronavirus is inducing or the pathogen causing the Coronavirus disease 2019 (COVID-2019). As used herein, the Coronavirus peptide comprises any viral peptide having at least 20%, at least 40%, at least 60%, at least 80%, at least 90%, at least 95%, or at least 99% amino acid sequence identity to a homolog derived from a Coronavirus, or any value and range therebetween. Each possibility represents a separate embodiment of the invention.

In some embodiments, the avian pathogenic virus is selected from: Infectious bursal disease virus (IBDV), Infectious bronchitis virus (IBV), Reovirus, Influenza virus, Chicken anaemia virus (CAV), Newcastle disease virus (NDV), Marek’s disease virus (MDV), Egg drop syndrome (EDS) avian adenovirus, and haemorrhagic enteritis virus (HEV).

In some embodiments, when the immunogenic antigen of IBDV is VP2; of IBV is S1, N, or any combination thereof; of Reovirus is Sigma C; of Influenza virus is HA and/or neuraminidase; of CAV is VP1, VP2, or a combination thereof; of NDV is HN, F, or a combination thereof; of EDS is KNOB; and of HEV is KNOB.

In some embodiments, a polynucleotide molecule encoding the chimeric polypeptide of the invention is provided.

In some embodiments, the polynucleotide comprises the nucleic acid:

ATGGGCAACAAGGTGAAATGCTACGTTCTGTTCACCGCGCTGCTGAGCAG
CCTGTATGCGCACGGTGCGCCGCAGACCATTACCGAGCTGTGCAGCGAAT
ACCGTAACACCCAAATCTATACCATTAACGACAAGATCCTGAGCTACACC
GAGAGCATGGCGGGCAAGCGTGAAATGGTGATCATTACCTTCAAAAGCGG
TGAAACCTTTCAGGTGGAAGTTCCGGGCAGCCAGCACATCGACAGCCAAA
AGAAAGCGATTGAGCGTATGAAAGATACCCTGCGTATCACCTATCTGACC
GAAACCAAGATTGACAAACTGTGCGTGTGGAACAACAAGACCCCGAACAG
CATCGCGGCGATTAGCATGAAAAACGGTGGCAGCGGTGGCAACAACACCG
CGAGCTGGTTCACCGCGCTGACCCAGCACGGTAAAGAGGACCTGAAATTT
CCGCGTGGTCAAGGCGTTCCGATCAACACCAACAGCAGCCCGGACGATCA
AATTGGTTACTATCGTCGTGCGACCCGTCGTATCCGTGGTGGCGACGGCA
AGATGAAGGACCTGAGCCCGCGTTGGTACTTCTACTACCTGGGCACCGGT
CCGGAGGCGGGTCTGCCGTATGGCGCGAACAAGGACGGTATCATTTGGGT
GGCGACCGAAGGCGCGCTGAACACCCCGAAAGATCACATTGGCACCCGTA
ACCCGGCGAACAACGCGGCGATCGTTCTGCAACTGCCGCAAGGCACCACC
CTGCCGAAGGGTTTTTACGCGGAAGGCAGC (SEQ ID NO:15).

In some embodiments, the polynucleotide comprises the nucleic acid:

ATGGGCAACAAGGTGAAATGCTACGTTCTGTTCACCGCGCTGCTGAGCAG
CCTGTATGCGCACGGTGCGCCGCAGACCATCACCGAGCTGTGCAGCGAAT
ACCGTAACACCCAAATCTATACCATTAACGACAAGATCCTGAGCTACACC
GAGAGCATGGCGGGCAAGCGTGAAATGGTGATCATTACCTTCAAAAGCGG
TGAAACCTTTCAGGTGGAAGTTCCGGGCAGCCAGCACATCGACAGCCAAA
AGAAAGCGATTGAGCGTATGAAAGATACCCTGCGTATCACCTATCTGACC
GAAACCAAGATTGATAAACTGTGCGTGTGGAACAACAAGACCCCGAACAG
CATCGCGGCGATTAGCATGAAAAACGGTGGCAGCGGTGGCGCGGCGGAGG
CGAGCAAGAAACCGCGTCAGAAGCGTACCGCGACCAAAGCGTACAACGTT
ACCCAAGCGTTCGGTCGTCGTGGTCCGGAGCAGACCCAGGGTAACTTTGG
CGACCAGGAACTGATCCGTCAAGGCACCGATTATAAGCACTGGCCGCAGA
TTGCGCAGTTTGCGCCGAGCGCGAGCGCGTTCTTTGGTATGAGCCGTATC
GGCATGGAAGTGACCCCGAGCGGCACCTGGCTGACCTACACCGGCGCGAT
TAAGCTGGACGATAAAGACCCGAACTTTAAAGATCAAGTTATCCTGCTGA
ACAAGCACATTGACGCGTATAAAACC (SEQ ID NO: 16).

In some embodiments, the polynucleotide encoding the chimeric polypeptide of the invention, a viral protein, a non-conjugated LTB, or any combination thereof, all as described herein, comprises a nucleic acid optimized for expression in an E. coli cell.

Methods for modifying the sequence of a polynucleotide so as to optimize its expression (e.g., according to codon preference of the expressing cell) in E. coli are common and would be apparent to one of ordinary skill in the art.

In some embodiments, an expression vector comprising the herein disclosed polynucleotide molecule is provided.

Methods for any one of: obtaining, manipulating, and optimizing an expression vector comprising a polynucleotide are well known in the art and are further elaborated herein below.

In some embodiments, there is provided a polynucleotide encoding the nucleocapsid protein comprising the nucleic acid sequence:

ATGGGCAACAAGGTGAAATGCTACGTTCTGTTCACCGCGCTGCTGAGCAG
CCTGTATGCGCACGGTGCGCCGCAGACCATTACCGAGCTGTGCAGCGAAT
ACCGTAACACCCAAATCTATACCATTAACGACAAGATCCTGAGCTACACC
GAGAGCATGGCGGGCAAGCGTGAAATGGTGATCATTACCTTCAAAAGCGG
TGAAACCTTTCAGGTGGAAGTTCCGGGCAGCCAGCACATCGACAGCCAAA
AGAAAGCGATTGAGCGTATGAAAGATACCCTGCGTATCACCTATCTGACC
GAAACCAAGATTGACAAACTGTGCGTGTGGAACAACAAGACCCCGAACAG
CATCGCGGCGATTAGCATGAAAAACGGTGGCAGCGGTGGCAACAACACCG
CGAGCTGGTTCACCGCGCTGACCCAGCACGGTAAAGAGGACCTGAAATTT
CCGCGTGGTCAAGGCGTTCCGATCAACACCAACAGCAGCCCGGACGATCA
AATTGGTTACTATCGTCGTGCGACCCGTCGTATCCGTGGTGGCGACGGCA
AGATGAAGGACCTGAGCCCGCGTTGGTACTTCTACTACCTGGGCACCGGT
CCGGAGGCGGGTCTGCCGTATGGCGCGAACAAGGACGGTATCATTTGGGT
GGCGACCGAAGGCGCGCTGAACACCCCGAAAGATCACATTGGCACCCGTA
ACCCGGCGAACAACGCGGCGATCGTTCTGCAACTGCCGCAAGGCACCACC
CTGCCGAAGGGTTTTTACGCGGAAGGCAGCCTCGAGCACCACCACCACCA
CCAC (SEQ ID NO: 27).

In some embodiments, there is provided a polynucleotide encoding the nucleocapsid protein comprising the nucleic acid sequence:

ATGGGCAACAAGGTGAAATGCTACGTTCTGTTCACCGCGCTGCTGAGCAG
CCTGTATGCGCACGGTGCGCCGCAGACCATCACCGAGCTGTGCAGCGAAT
ACCGTAACACCCAAATCTATACCATTAACGACAAGATCCTGAGCTACACC
GAGAGCATGGCGGGCAAGCGTGAAATGGTGATCATTACCTTCAAAAGCGG
TGAAACCTTTCAGGTGGAAGTTCCGGGCAGCCAGCACATCGACAGCCAAA
AGAAAGCGATTGAGCGTATGAAAGATACCCTGCGTATCACCTATCTGACC
GAAACCAAGATTGATAAACTGTGCGTGTGGAACAACAAGACCCCGAACAG
CATCGCGGCGATTAGCATGAAAAACGGTGGCAGCGGTGGCGCGGCGGAGG
CGAGCAAGAAACCGCGTCAGAAGCGTACCGCGACCAAAGCGTACAACGTT
ACCCAAGCGTTCGGTCGTCGTGGTCCGGAGCAGACCCAGGGTAACTTTGG
CGACCAGGAACTGATCCGTCAAGGCACCGATTATAAGCACTGGCCGCAGA
TTGCGCAGTTTGCGCCGAGCGCGAGCGCGTTCTTTGGTATGAGCCGTATC
GGCATGGAAGTGACCCCGAGCGGCACCTGGCTGACCTACACCGGCGCGAT
TAAGCTGGACGATAAAGACCCGAACTTTAAAGATCAAGTTATCCTGCTGA
ACAAGCACATTGACGCGTATAAAACCCTCGAGCACCACCACCACCACCAC
(SEQ ID NO: 29).

### Cells

In some embodiments, there is provided a cell of the invention comprising: (a) the chimeric polypeptide of the invention; (b) the herein disclosed polynucleotide molecule; (c) the herein disclosed expression vector; or any combination of (a) to (c).

In some embodiments, the cell of the invention comprises a chimeric polypeptide comprising a plurality of viral peptides, e.g., a viral fusion peptide. In one embodiment, the cell comprises a chimeric polypeptide comprising the LTB or CTB polypeptide, the spike polypeptide, and the nucleocapsid protein. In some embodiments, the cell comprises a plurality of chimeric polypeptides. In some embodiments, the cell comprises a first chimeric polypeptide comprising the LTB or CTB polypeptide and a first viral peptide, and a second chimeric polypeptide comprising the LTB or CTB polypeptide and a second viral peptide. In one embodiment, the cell comprises a first chimeric polypeptide comprising the LTB or CTB polypeptide and a viral spike peptide, and a second chimeric polypeptide comprising the LTB or CTB polypeptide and a nucleocapsid protein.

In some embodiments, the cell is a naive cell or a recombinant cell.

In some embodiments, the cell further lacks one or more extra-cellular proteases.

In some embodiments, the cell is a bacterial cell. In some embodiments, the cell is a plant cell. In some embodiments, the cell is a mammalian cell. In some embodiments, the cell is an insect cell. In some embodiment, the cell is a yeast cell. In some embodiments, the cell is an E. coli cell. In some embodiments, the cell further comprises a GSP operon encoding a type II secretion system; and optionally lacks a polynucleotide comprising a gene encoding transcription factor HNS. In some embodiments, the cell is an E. coli cell derived from a naive (non-recombinant) E. coli K12 or ER2566.

In some embodiments, the cell is used for or in production of the chimeric polypeptide of the invention, e.g., as a recombinant protein expression system. In some embodiments, the cell is transformed with a chimeric polynucleotide encoding the chimeric polypeptide of the invention. In some embodiments, the cell is a secreting cell. In some embodiments, the cell is cultured in a medium. In some embodiments, the medium is a culture/growth medium. In some embodiments, the cell is cultured under sufficient conditions so as to express and secrete the chimeric polypeptide of the invention. In some embodiments, the chimeric polypeptide is retrieved from the growth/culture medium. Methods for expressing recombinant proteins in a cell expression system and purification of same are common and would be apparent to one of ordinary skill in the art and are further elaborated hereinbelow.

In some embodiments, the chimeric polypeptide of the invention comprises a linker. As used herein, the term “linker” refers a molecule or macromolecule serving to connect different moieties of a peptide or a polypeptide. In one embodiment, said linker may also facilitated other functions, including, but not limited to, preserving biological activity, maintaining sub-units and domains interactions, and others. Expressing of a gene within a cell is well known to one skilled in the art. It can be carried out by, among many methods, transfection, viral infection, or direct alteration of the cell’s genome. In some embodiments, the gene is in an expression vector such as plasmid or viral vector. One such example of an expression vector containing p16-Ink4a is the mammalian expression vector pCMV p16 INK4A available from Addgene.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the gene is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyses the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighbouring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat sock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation, and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

A person with skill in the art will appreciate that a gene can also be expressed from a nucleic acid construct administered to the individual employing any suitable mode of administration, described hereinabove (i.e., in vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex vivo gene therapy).

### Methods

In some embodiments, a method for treating or preventing a viral infection in a subject in need thereof, comprising administering to the subject a therapeutically effective amount of the herein disclosed pharmaceutical composition, thereby treating, or preventing a viral infection in the subject, is provided.

In some embodiments, administering is by a route selected from: oral, transdermal, anal, nasal, topical, and in-ovo.

In some embodiments, a method for producing a vaccine, comprising: (a) obtaining a chimeric polypeptide comprising LTB and a viral polypeptide, wherein the LTB and the viral polypeptide are operably linked, or a cell comprising the chimeric polypeptide, contacting a host subject with the chimeric polypeptide or with the cell comprising thereof, determining the presence of immunoglobulin G (IgG) targeting the chimeric polypeptide in a sample of the host subject, and selecting at least one chimeric polypeptide or a cell comprising thereof, that substantially induced IgG production in the host subject; or (b) culturing a host cell comprising one or more expression vectors comprising a nucleic acid sequence encoding a chimeric polypeptide, wherein the nucleic acid sequence is that of a chimeric polypeptide that was selected by: i. obtaining a chimeric polypeptide comprising LTB and a viral polypeptide, wherein the LTB and the viral polypeptide are operably linked; ii. contacting a host subject with the chimeric polypeptide iii. determining the presence of immunoglobulin G (IgG) targeting the chimeric polypeptide in a sample of the host subject; and iv. selecting at least one chimeric polypeptide that substantially induced IgG production in the host subject; thereby producing a vaccine.

As used herein, substantially is compared to control. In some embodiments, a control is a non-immunogenic peptide or antigen. In some embodiments, a control does not induce immunoglobulin production. In some embodiments, a control does not induce production of immunoglobulin targeting the chimeric polypeptide of the invention.

### Definitions

The term “amino acid” as used herein means an organic compound containing both a basic amino group and an acidic carboxyl group.

The term “amino acid residue” as used herein refers to the portion of an amino acid that is present in a peptide.

The term “peptide bond” means a covalent amide linkage formed by loss of a molecule of water between the carboxyl group of one ammo acid and the ammo group of a second ammo acid.

The terms “peptide”, “polypeptide” and “protein” as used herein encompass native peptides, peptidomimetics (typically including non-peptide bonds or other synthetic modifications) and the peptide analogs peptoids and semi-peptoids or any combination thereof. In another embodiment, the terms “peptide”, “polypeptide” and “protein” apply to amino acid polymers in which at least one amino acid residue is an artificial chemical analog of a corresponding naturally occurring amino acid.

The term “peptide”, “polypeptide” and “protein” are used herein interchangeably.

One of skill in the art will recognize that individual substitutions, deletions or additions to a peptide, or protein sequence which alters, adds, or deletes a single amino acid or a small percentage of amino acids in the encoded sequence is a conservatively modified variant where the alteration results in the substitution of an amino acid with a similar charge, size, and/or hydrophobicity characteristics, such as, for example, substitution of a glutamic acid (E) to an aspartic acid (D).

As used herein, the phrase “conservative substitution” also includes the use of a chemically derivatized residue in place of a non-derivatized residue provided that such peptide displays the requisite function as specified herein.

Peptide derivatives can also include side chain bond modifications, including but not limited to -CH2-NH-, -CH2-S-, -CH2-S=0, OC-NH-, -CH2-O-, -CH2-CH2-, S=C-NH-, and -CH=CH-, and backbone modifications such as modified peptide bonds. Peptide bonds (-CO-NH-) within the peptide can be substituted, for example, by N-methylated bonds (-N(CH3)-CO-); ester bonds (-C(R)H-C-O-O-C(R)H-N); ketomethylene bonds (-CO-CH2-); a-aza bonds (-NH-N(R)-CO-), wherein R is any alkyl group, e.g., methyl; carba bonds (-CH2-NH-); hydroxyethylene bonds (-CH(OH)-CH2-); thioamide bonds (-CS-NH); olefmic double bonds (-CH=CH-); and peptide derivatives (-N(R)-CH2-CO-), wherein R is the “normal” side chain, naturally presented on the carbon atom. These modifications can occur at one or more of the bonds along the peptide chain and even at several (e.g., 2-3) at the same time.

### Peptide Synthesis

According to one embodiment, the peptide of the invention may be synthesized or prepared by any method and/or technique known in the art for peptide synthesis.

According to another embodiment, the peptide may be synthesized by a solid phase peptide synthesis method of Merrifield (see J. Am. Chem. Soc, 85:2149, 1964). According to another embodiment, the peptide of the invention can be synthesized using standard solution methods, which are well known in the art (see, for example, Bodanszky, M., Principles of Peptide Synthesis, Springer- Verlag, 1984).

In general, the synthesis methods comprise sequential addition of one or more amino acids or suitably protected amino acids to a growing peptide chain bound to a suitable resin. Normally, either the amino or carboxyl group of the first amino acid is protected by a suitable protecting group. The protected or derivatized amino acid can then be either attached to an inert solid support (resin) or utilized in solution by adding the next amino acid in the sequence having the complimentary (amino or carboxyl) group suitably protected, under conditions conductive for forming the amide linkage. The protecting group is then removed from this newly added amino acid residue and the next amino acid (suitably protected) is added, and so forth. After all the desired amino acids have been linked in the proper sequence, any remaining protecting groups are removed sequentially or concurrently, and the peptide chain, if synthesized by the solid phase method, is cleaved from the solid support to afford the final peptide.

In the solid phase peptide synthesis method, the alpha-amino group of the amino acid is protected by an acid or base sensitive group. Such protecting groups should have the properties of being stable to the conditions of peptide linkage formation, while being readily removable without destruction of the growing peptide chain. Suitable protecting groups are t-butyloxycarbonyl (BOC), benzyloxycarbonyl (Cbz), biphenylisopropyloxycarbonyl, t-amyloxycarbonyl, isobornyloxycarbonyl, (alpha, alpha)-dimethyl-3 ,5 dimethoxybenzyloxycarbonyl, o-nitrophenylsulfenyl, 2-cyano-t-butyloxycarbonyl, 9-fluorenylmethyloxycarbonyl (Fmoc) and the like. In the solid phase peptide synthesis method, the C-terminal amino acid is attached to a suitable solid support. Suitable solid supports useful for the above synthesis are those materials, which are inert to the reagents and reaction conditions of the stepwise condensation-deprotection reactions, as well as being insoluble in the solvent media used. Suitable solid supports are chloromethylpolystyrene-divinylbenzene polymer, hydroxymethyl-polystyrene-divinylbenzene polymer, and the like. The coupling reaction is accomplished in a solvent such as ethanol, acetonitrile, N,N-dimethylformamide (DMF), and the like. The coupling of successive protected amino acids can be carried out in an automatic peptide synthesizer as is well known in the art.

In another embodiment, a peptide of the invention may be synthesized such that one or more of the bonds, which link the amino acid residues of the peptide are non-peptide bonds. In another embodiment, the non-peptide bonds include, but are not limited to, imino, ester, hydrazide, semicarbazide, and azo bonds, which can be formed by reactions well known to one skilled in the art.

The invention further encompasses a polynucleotide sequence comprising a nucleic acid encoding any of the peptides of the invention. In another embodiment, the nucleic acid sequence encoding the peptide is at least 70%, or alternatively at least 80%, or alternatively at least 90%, or alternatively at least 95%, or alternatively at least 99% homologous to the nucleic acid sequence encoding the nucleic acid sequence of the peptides of the invention or a derivative thereof, or any value and range therebetween. Each possibility represents a separate embodiment of the invention.

In some embodiment, the invention provides a polynucleotide encoding the peptide of the invention. In some embodiments, the invention provides a polynucleotide encoding the chimera of the invention.

In some embodiments, a polynucleotide molecule encodes a peptide comprising non-canonical amino acids.

In some embodiments, the polynucleotide of the invention is ligated into an expression vector, comprising a transcriptional control of a cis-regulatory sequence (e.g., promoter sequence). In some embodiments, the cis-regulatory sequence is suitable for directing constitutive expression of the peptide of the invention. In some embodiments, the cis-regulatory sequence is suitable for directing tissue-specific expression of the peptide of the invention. In some embodiments, the cis-regulatory sequence is suitable for directing inducible expression of the peptide of the invention.

The term “polynucleotide” refers to a nucleic acid (e.g., DNA or RNA) sequence that comprises coding sequences necessary for the production of a peptide. In one embodiment, a polynucleotide refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

In one embodiment, “complementary polynucleotide sequence” refers to a sequence, which results from reverse transcription of messenger RNA using a reverse transcriptase or any other RNA-dependent DNA polymerase. In one embodiment, the sequence can be subsequently amplified in vivo or in vitro using a DNA polymerase.

In one embodiment, “genomic polynucleotide sequence” refers to a sequence derived or isolated from a chromosome and, thus it represents a contiguous portion of a chromosome.

In one embodiment, “composite polynucleotide sequence” refers to a sequence, which is at least partially complementary and at least partially genomic. In one embodiment, a composite sequence can include some exonal sequences required to encode the peptide of the invention, as well as some intronic sequences interposing therebetween. In one embodiment, the intronic sequences can be of any source, including of other genes, and typically may include conserved splicing signal sequences. In one embodiment, intronic sequences include cis-acting expression regulatory elements.

In some embodiments, a polynucleotide of the invention is prepared using PCR techniques, or any other method or procedure known to one of ordinary skill in the art.

In some embodiments, an expression vector comprising a polynucleotide encoding the peptide of the invention or a chimera comprising the same, is provided.

In one embodiment, a polynucleotide of the invention is inserted into expression vectors (i.e., a nucleic acid construct) to enable expression of a recombinant peptide. In one embodiment, the expression vector includes additional sequences which render this vector suitable for replication and integration in prokaryotes. In one embodiment, the expression vector includes additional sequences which render this vector suitable for replication and integration in eukaryotes. In one embodiment, the expression vector includes a shuttle vector which renders this vector suitable for replication and integration in both prokaryotes and eukaryotes. In some embodiments, cloning vectors comprise transcription and translation initiation sequences (e.g., promoters, enhancers) and transcription and translation terminators (e.g., polyadenylation signals).

In some embodiments, a cell comprising any one of: the peptide of the invention; a chimera comprising the same; a polynucleotide encoding the peptide of the invention; and an expression vector comprising the polynucleotide encoding the peptide of the invention, is provided.

In one embodiment, a variety of prokaryotic or eukaryotic cells can be used as host-expression systems to express the peptide of the invention. In some embodiments, these include, but are not limited to, microorganisms, such as bacteria transformed with a recombinant bacteriophage DNA, plasmid DNA or cosmid DNA expression vector containing the peptide coding sequence; yeast transformed with recombinant yeast expression vectors containing the peptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the peptide coding sequence.

In some embodiments, non-bacterial expression systems are used (e.g., mammalian expression systems) to express the peptide of the invention. In one embodiment, the expression vector is used to express the polynucleotide of the invention in mammalian cells.

In some embodiments, in bacterial systems, a number of expression vectors can be advantageously selected depending upon the use intended for the peptide expressed. In one embodiment, large quantities of peptide are desired. In one embodiment, vectors that direct the expression of high levels of the protein product, possibly as a fusion with a hydrophobic signal sequence, which directs the expressed product into the periplasm of the bacteria or the culture medium where the protein product is readily purified are desired. In one embodiment, certain fusion protein engineered with a specific cleavage site to aid in recovery of the peptide. In one embodiment, vectors adaptable to such manipulation include, but are not limited to, the pET series of E. coli expression vectors [Studier et al., Methods in Enzymol. 185:60-89 (1990)].

In one embodiment, yeast expression systems are used. In one embodiment, a number of vectors containing constitutive or inducible promoters can be used in yeast as disclosed in U.S. Pat. No. 5,932,447. In another embodiment, vectors which promote integration of foreign DNA sequences into the yeast chromosome are used.

In one embodiment, the expression vector may further include additional polynucleotide sequences that allow, for example, the translation of several proteins from a single mRNA such as an internal ribosome entry site (IRES).

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses can be used. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression of the peptide of the invention. In one embodiment, lateral infection is inherent in the life cycle of, for example, retrovirus and is the process by which a single infected cell produces many progeny virions that bud off and infect neighbouring cells. In one embodiment, the result is that a large area becomes rapidly infected, most of which was not initially infected by the original viral particles. In one embodiment, the viral vectors that are produced are unable to spread laterally. In one embodiment, this characteristic can be useful if the desired purpose is to introduce a specified gene into only a localized number of targeted cells.

Various methods can be used to introduce an expression vector into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a peptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation, and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the peptide), the expression construct can also include sequences engineered to optimize stability, production, purification, yield, or activity of the expressed peptide.

In some embodiments, transformed cells are cultured under effective conditions, which allow for the expression of high amounts of a recombinant peptide. In some embodiments, effective culture conditions include, but are not limited to, effective media, bioreactor, temperature, pH, and oxygen conditions that permit protein production. In one embodiment, an effective medium refers to any medium in which a cell is cultured to produce a recombinant peptide of the present invention. In some embodiments, a medium typically includes an aqueous solution having assimilable carbon, nitrogen and phosphate sources, and appropriate salts, minerals, metals, and other nutrients, such as vitamins. In some embodiments, the cells can be cultured in conventional fermentation bioreactors, shake flasks, test tubes, microtiter dishes and petri plates. In some embodiments, culturing is carried out at a temperature, pH, and oxygen content appropriate for a recombinant cell. In some embodiments, culturing conditions are within the expertise of one of ordinary skill in the art.

In some embodiments, depending on the vector and host system used for production, resultant peptide of the invention either remains within the recombinant cell, secreted into the fermentation medium, secreted into a space between two cellular membranes, such as the periplasmic space in E. coli; or retained on the outer surface of a cell or viral membrane. In one embodiment, following a predetermined time in culture, recovery of the recombinant peptide is affected.

In one embodiment, the phrase “recovering the recombinant peptide” as used herein, refers to collecting the whole fermentation medium containing the peptide and need not imply additional steps of separation or purification.

In one embodiment, a peptide of the invention is purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

In some embodiments, the purified chimeric peptide of the invention is further used according to methods disclosed herein below, e.g., for treating or preventing an infectious disease in a subject in need thereof, for vaccinating of a subject in need thereof, etc.

In one embodiment, to facilitate recovery, the expressed coding sequence can be engineered to encode the peptide of the invention and fused cleavable moiety. In one embodiment, a fusion protein can be designed so that the peptide can be readily isolated by affinity chromatography, e.g., by immobilization on a column specific for the cleavable moiety. In one embodiment, a cleavage site is engineered between the peptide and the cleavable moiety, and the peptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

In one embodiment, the chimeric peptide of the invention is retrieved in “substantially pure” form that allows for the effective use of the protein in the applications described herein. In one embodiment, the chimeric peptide of the invention is retrieved partially pure. As used herein, “partially” refers to any integer of 99% or less. In one embodiment, partially pure refers to any case wherein the chimeric peptide of the invention is retrieved with at least one component of the culture wherein the chimeric peptide of the invention was produced, e.g., a cell, cell debris, cell culture media, growth media, a nutrient, a metabolite, etc. Methods for determining the purity level of a retrieved peptide are common and would be apparent to one of ordinary skill in the art. Non-limiting examples for such methods, include but are not limited to, protein gel electrophoresis, western blot, ELISA, mass spectrometry, e.g., MS-MS, HPLC, MALDI-TOF, and others.

As used herein, the term “substantially pure” describes a peptide or other material which has been separated from its native contaminants. Typically, a monomeric peptide is substantially pure when at least about 60 to 75% of a sample exhibits a single peptide backbone. Minor variants or chemical modifications typically share the same peptide sequence. A substantially pure peptide can comprise over about 85 to 90% of a peptide sample, and can be over 95% pure, over 97% pure, or over about 99% pure, or any value and range therebetween. Each possibility represents a separate embodiment of the invention. Purity can be measured on a polyacrylamide gel, with homogeneity determined by staining. Alternatively, for certain purposes high resolution may be necessary and HPLC or a similar means for purification can be used. For most purposes, a simple chromatography column or polyacrylamide gel can be used to determine purity.

The term “purified” does not require the material to be present in a form exhibiting absolute purity, exclusive of the presence of other compounds. Rather, it is a relative definition. A peptide is in the “purified” state after purification of the starting material or of the natural material by at least one order of magnitude, 2 or 3, or 4 or 5 orders of magnitude.

In one embodiment, the peptide of the invention is substantially free of naturally associated host cell components. The term “substantially free of naturally-associated host cell components” describes a peptide or other material which is separated from the native contaminants which accompany it in its natural host cell state. Thus, a peptide which is chemically synthesized or synthesized in a cellular system different from the host cell from which it naturally originates will be free from its naturally associated host cell components.

In one embodiment, the peptide of the invention can also be synthesized using in vitro expression systems. In one embodiment, in vitro synthesis methods are well known in the art and the components of the system are commercially available. Non-limited example for in vitro system includes, but is not limited to in vitro translation, such as exemplified herein below.

As used herein, the term “chimera” encompasses any conjugate comprising two or more moieties, wherein the two or more moieties are bound to one another either directly or indirectly, and wherein the moieties are either derived from distinct origins or are not naturally bound to one another. In some embodiments, the two or more moieties have: distinct functions, originate or derived from different genes, peptides, genomic regions, or species, distinct chemical classification (e.g., a peptide and a polynucleotide, as exemplified herein).

In some embodiments, the chimera of the invention comprises the peptide of the invention bound directly or indirectly to an agent, wherein the agent is selected from: a nucleotide, an oligonucleotide, a polynucleotide, an amino acid, a peptide, a peptide, a protein, a small molecule, a synthetic molecule, an organic molecule, an inorganic molecule, a polymer, a synthetic polymer, or any combination thereof.

As used herein, the term “directly” refers to cases wherein the peptide of the invention is bound to the agent in a covalent bond.

As used herein, the term “indirectly” refers to cases wherein each of the peptide of the invention and the agent are bound to a linker or a spacing element and not directly to one another. In some embodiments, the peptide is covalently bound to the linker. In some embodiments, the agent is either covalently or non-covalently bound to the linker.

As used herein, the term “covalent bond” refers to any bond which comprises or involves electron sharing. Non-limiting examples of a covalent bond include, but are not limited to peptide bond, glyosidic bond, ester bond, phosphor diester bond.

As used herein, the term “non-covalent bond” encompasses any bond or interaction between two or more moieties which do not comprise or do not involve electron sharing. Non-limiting examples of a non-covalent bond or interaction include, but are not limited to, electrostatic, π-effect, van der Waals force, hydrogen bonding, and hydrophobic effect.

The term “linker” refers to a molecule or macromolecule serving to connect different moieties of the chimera, that is the peptide of the invention and the agent. In one embodiment, a linker may also facilitate other functions, including, but not limited to, preserving biological activity, maintaining sub-units and domains interactions, and others.

In another embodiment, a linker may be a monomeric entity such as a single amino acid. In another embodiment, amino acids with small side chains are especially preferred, or a peptide chain, or polymeric entities of several amino acids. In another embodiment, a peptide linker is 2 to 30 amino acids long, 2 to 25 amino acids long, 4 to 23 amino acids long, 4 to 20 amino acids long, 5 to 22 amino acids long, or 2 to 28 amino acids long. Each possibility represents a separate embodiment of the invention. In another embodiment, a peptide linker is at least 6 amino acids long, at least 8 amino acids long, at least 10 amino acids long, at least 12 amino acids long, at least 15 amino acids long, at least 17 amino acids long, at least 20 amino acids long, at least 22 amino acids long, at least 25 amino acids long, at least 27 amino acids long, or at least 30 amino acids long, or any value and range therebetween. Each possibility represents a separate embodiment of the invention. In one embodiment, a linker may be a nucleic acid encoding a small peptide chain. In another embodiment, a linker encodes a peptide linker of 6 to 30 amino acids long, 6 to 25 amino acids long, 7 to 23 amino acids long, 8 to 20 amino acids long, 10 to 22 amino acids long, or 12 to 28 amino acids long. Each possibility represents a separate embodiment of the invention. In another embodiment, a linker encodes a peptide linker of at least 6 amino acids long, at least 8 amino acids long, at least 10 amino acids long, at least 12 amino acids long, at least 15 amino acids long, at least 17 amino acids long, at least 20 amino acids long, at least 22 amino acids long, at least 25 amino acids long, at least 27 amino acids long, or at least 30 amino acids long, or any value and range therebetween. Each possibility represents a separate embodiment of the invention.

In some embodiments, a peptide of the invention and a peptide linker are transcribed from a single polynucleotide sequence. In some embodiments, the peptide of the invention and a peptide linker are transcribed from a single polynucleotide sequence so as to provide the chimera of the invention. In some embodiments, the peptide of the invention and the peptide linker reside within a single peptide chain. In some embodiments, the peptide of the invention and the peptide linker are adjacent to one another in a manner that the last amino acid at the C′ terminus of the peptide of the invention is bound via a peptide bond to the first amino acid of the N′ terminus of the peptide linker. In some embodiments, the peptide of the invention and the peptide linker are adjacent to one another in a manner that the first amino acid at the N′ terminus of the peptide of the invention is bound via a peptide bond to the last amino acid of the C′ terminus of the peptide linker.

In some embodiments, the peptide of the invention may be attached or linked to an agent via a chemical linker. Chemical linkers are well known in the art and include, but are not limited to, dicyclohexylcarbodiimide (DCC), N-hydroxysuccinimide (NHS), maleiimidobenzoyl-N-hydroxysuccinimide ester (MBS), N-ethyloxycarbonyl-2-ethyloxy-1,2-dihydroquinoline (EEDQ), N-isobutyloxy-carbonyl-2-isobutyloxy-1,2-dihydroquinoline (IIDQ).

Recombinant technology may be used to express the peptide of the invention, and is well known in the art. In another embodiment, the linker may be a cleavable linker, resulting in cleavage of the peptide of the invention once delivered to the tissue or cell of choice. In such an embodiment, the cell or tissue would have endogenous (either naturally occurring enzyme or be recombinantly engineered to express the enzyme) or have exogenous (e.g., by injection, absorption, or the like) enzyme capable of cleaving the cleavable linker.

In another embodiment, the linker may be biodegradable such that the peptide of the invention is further processed by hydrolysis and/or enzymatic cleavage inside cells. In some embodiments, a readily cleavable group include acetyl, trimethylacetyl, butanoyl, methyl succinoyl, t-butyl succinoyl, ethoxycarbonyl, methoxycarbonyl, benzoyl, 3-aminocyclohexylidenyl, and the like.

In some embodiments, a peptide linker has an electric charge at a pH ranging from 6.5 to 8.

In some embodiments, the linker has a positive electric charge. In some embodiments, the linker has a negative electric charge.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising,” indicate the inclusion of any recited integer or group of integers but not the exclusion of any other integer or group of integers.

As used herein, the term “consists essentially of′, or variations such as “consist essentially of′ or “consisting essentially of′ as used throughout the specification and claims, indicate the inclusion of any recited integer or group of integers, and the optional inclusion of any recited integer or group of integers that do not materially change the basic or novel properties of the specified method, structure, or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, and “having” are interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

As used herein, the term “about” when combined with a value refers to plus and minus 10% of the reference value. For example, a length of about 1000 nanometres (nm) refers to a length of 1000 nm ± 100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, CT (1994); Mishell and Shiigi (eds), “Strategies for Protein Purification and Characterization - A Laboratory Course Manual” CSHL Press (1996); all of which are incorporated by reference. Other general references are provided throughout this document.

### Materials and Methods

### Cloning Description

The backbone vector is a pET28a(+) plasmid from Novagen (Merck). The pET-28a-c(+) vectors carry an N-terminal His•Tag®/thrombin/T7•Tag® configuration plus an optional C-terminal His•Tag sequence. The vector is composed of 5,369 bp.

Three plasmids were constructed from the aforementioned backbone by using restriction enzymes and ligation. This cloning strategy removes all N-terminal tags from the vector; one for each chimeric protein which will serve as a Drug Substance.

p28L-NN (corresponds to the N′ terminus of a nucleocapsid protein, and p28L-NC corresponds to the C′ terminus of a nucleocapsid protein) are circular DNA bacterial plasmids, 6016, 5962 bp (base pairs) in length respectively, that carries the gene encoding the LTB-NN and LTB-NC chimeric proteins, respectively.

LTB-NN and LTB-NC insert into the vector was performed by PCR amplification of synthetic liner construct made by IDT-DNA followed by restriction digestion of the PCR products and the vector DNA with NcoI and XhoI (Thermo) and ligation.

### Insert Amplification and Plasmid Construction

Oligonucleotides encoding LTB, LTB-NN and LTB-NC genes were ordered from IDT DNA. In order to facilitate cloning of the PCR product, NcoI and XhoI enzyme restriction sites were incorporated into the PCR primer design. The resulting PCR product was digested with the NcoI and XhoI enzymes and ligated into the NcoI and XhoI sites of the pET28a(+), yielding the p28LTB, p28L-NN, and p28L-NC.

### Transformation

E. coli C41 (Lucigen) was transformed by Electro-transformation. Competent C41 cells transferred from -80° C. to ice for slow thawing. Electro-transformation cuvettes were also placed in ice.

Four (4) µl of each suspended ligation mixture was added to 100 µl competent cells and transferred to a pre chilled cuvettes. BIO-RAD MicroPulser™ was used to pulse the cells with the “Ec1” setting. Cells were resuspended with fresh 900 µl LB medium and transferred to culture tubes for recovery at 37° C. for 1 h at 200 rpm. Cells were spread as is and diluted × 100 on LB-agar plates supplemented with 50 µg/ml kanamycin and 1% glucose. Plates were incubated at 37° C. O.N for colony formation.

### Clonal Selection

Single positive E. coli colonies were selected and analyzed by Colony PCR with specific primers plasmid purification, and nucleotide sequencing.

### Inoculation

Starters were grown with LB (10 g/l bacto tryptone, 5 g/l yeast extract) + 1% glucose, 100 µg/ml Kanamycin, at Starters/ flask volume ratio: 1/100.

### Main Growth and Induction

Cells were grown with 2×YT Medium (16 g/l bacto tryptone, 10 g/l yeast extract, 5 g/l NaCl), containing 100 µg/ml Kanamycin, at 37° C., 250 RPM until OD₆₀₀ reached 0.6. Growth temperature was lowered to 25° C. and after 20 minutes 0.4 mM IPTG was added. Cells were further grown for 16 hours at 25° C.

### Cell Lysis

Cells were collected using centrifugation: 8,000 RPM, 20 minutes, 4° C. and media and pellets were separated.

Cells were lysed using a homogenizer (at 19,000 PSI, Pass the sample ×3) with lysis buffer: 50 mM phosphate buffer pH 7.2, 300 mM NaCl, 0.1% Triton X100, 0.1% Tween 20, 1 tablet of protease inhibitor per 500 ml culture. Add eighty 80 ml/per 1 litre originated pellet.

Clarify the lysate by centrifugation (2 cycles of 15,000 g, 20 min, 4° C.). The supernatant of the lysate is separated from the pellet.

### Protein Purification

Protein was purified on a Pack BIO-RAD gravity column with 0.5 ml galactose-sepharose (Thermo) resin (50% slurry) for 500 ml culture. Column was equilibrate using binding buffer (50 mM Phosphate buffer pH 7.2, 300 mM NaCl, 0.1% Triton X-100, 0.1% Tween 20), clarified lysate loaded on the column. Washing was performed using wash buffer (50 mM Phosphate buffer pH 7.2, 300 mM NaCl, 0.1% Triton X-100, 0.1% Tween 20). Protein was eluted using elution buffer (50 mM Phosphate buffer pH 7.2, 300 mM NaCl, 0.1% triton X-100, 0.1% Tween 20, 100 mM Galactose).

Fractions were analyzed using SDS-PAGE and combine relevant fraction. Determine concentration by, SDS-PAGE, Bradford, and activity. Dialyze 3 times 1:100 against PBS and freeze samples at a concentration of 2-3 mg/ml after the addition of 15% glycerol, for NC and LTB. For NN add 0.5% tween 20 and dialyze against PBS+0.5% tween 20.

### Example 1

### Construction of LTB-Hypersecretion Strains of E. Coli

E. coli strain MG1655 contains silenced operon (GSP operon) which encodes for a type II secretion system. This system is responsible for the secretion of wild-type LTB protein. HNS is a regulator protein, which represses the GSP operon under standard growth conditions. Three strategies were used in order to increase the expression of the GSP system, in order to increase the overall secretion of LTB-fusion proteins. Mutations in the promoter region of the GSP operon were designed to strengthen the Sigma70 consensus sequence and weaken repressor binding sites. Over expression of the full GSP operon on a plasmid was performed. Deletion of the gene encoding HNS was performed as it was previously reported to increase GSP expression.

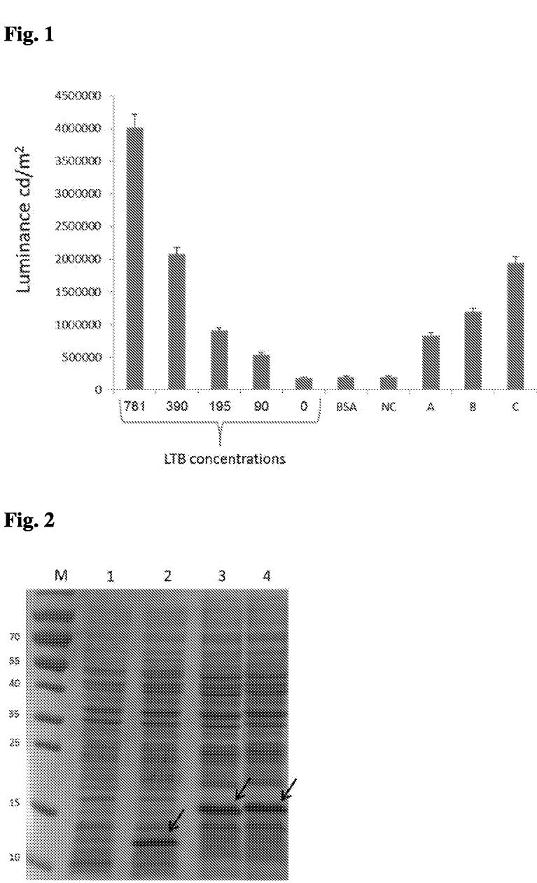

As shown in FIG. 1, secretion of natural LTB in the wildtype strain after 24 h incubation was about 200 ng/ml (comparable to reported results). Mutations in the promoter region increases the expression by ~50%, and a strain that over expressed the GSP operon and carried a deletion of the HNS gene showed about 2-fold more secreted LTB (compared to the WT) as determined by a standard GM1-binding ELISA assay that detect functional (receptor binding) LTB.

### Example 2

### Construction of LTB-N Fusion Proteins

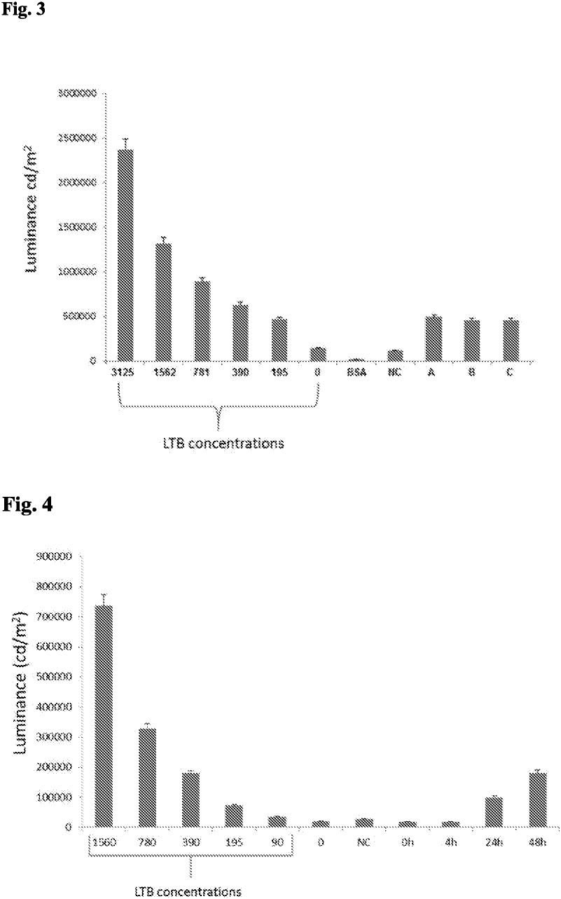

IBV contains two major proteins with known immunological relevance. The S (spike) protein and the N (nucleosome) protein were both shown to participate in induction of effective immune response in chickens. Two different fragments of the N protein were genetically fused to the LTB sequence using a 5 amino acid (AA) glycine rich linker. Amino acids 29-160 and amino acids 218-326 that were previously shown to form stable and soluble domains were chosen to represent the N protein. Expression of the LTB-N fusion proteins are visualized by a total protein Coomassie staining after separation of E. coli proteins by SDS-PAGE electrophoresis (FIG. 2). Strain expressing the WT LTB was shown as a control. ELISA assay for secreted LTB demonstrated that both fusion proteins were secreted with similar efficacy as the WT protein. Similarly, to the WT protein, introduction of the fusion construct into the hypersecretion strains, resulted in increased presence of LTB-fusion proteins in the growth medium (FIG. 3). Accumulation of the LTB-N fusion proteins in the growth medium was further increased after a 48 hr-incubation time (FIG. 4).

### Example 3

### Vaccination Study of Chickens With LTB-N Fusion Proteins

One (1) month old chickens were used to study induction of specific immune response against IBV N protein following oral intake of E. coli strains that secrete LTB-N fusion proteins. Eight groups were used in the first study: A: injection of PBS (control); B: injection of inactivated IBV virus; C: injection of WT LTB protein; D: injection of LTB-N 218-326; E: Oral intake of WT E. coli MG1655; F: Oral intake of E. coli, secreting WT LTB; G: oral intake of E. coli, secreting LTB-N 218-326; and H: oral intake of E. coli, secreting LTB-N 29-160. All potential vaccines and controls were administered twice, with two weeks interval, and then blood was collected from each chicken for analysis of serum antibodies. The sera of each group were reacted against four samples in a western blot analysis (1, Total proteins of WT bacteria; 2, Partially purified LTB; 3, Partially purified N 218-326; and 4, Partially purified N 29-160).

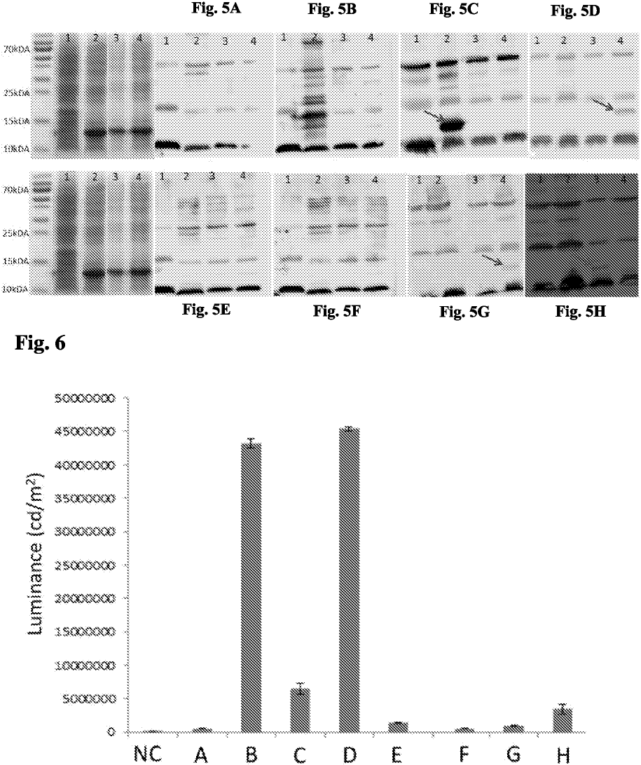

As shown in FIG. 5, both injected and orally administered vaccines, resulted in the presence of corresponding antibodies, indicating that LTB-fusion protein, secreted by bacteria, can engage with the immune system, and induce an immunogenic reaction. Interestingly, the LTB-N fusion proteins resulted in formation of antibodies against the N domain, while no reaction was observed against the highly immunogenic LTB carrier domain, in this analysis.

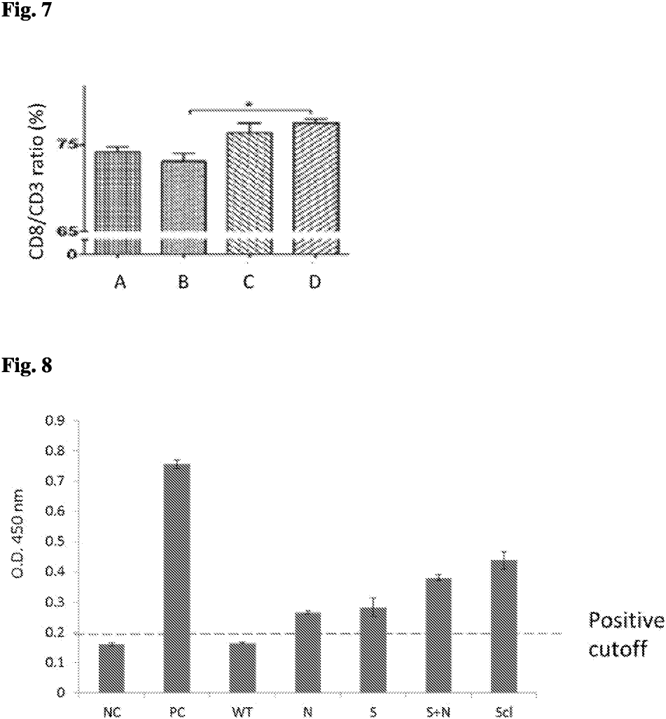

Examination of the sera in an ELISA assay, using full-length purified N protein, and against purified LTB, showed that the injected antigens produced stronger reactions, however, all orally administered antigens showed stronger signal than the controls (FIG. 6). Sera from orally administered LTB-N 218-326 showed the strongest signal against both LTB and N proteins. Representative chickens from each group of orally administered live bacteria, were sacrificed, and splenocytes activation potential was determined (as a measurement of cellular immunity) using FACS (fluorescence-activated cell sorting), by measuring the abundance of CD8 from the total CD3-T expressing cells, after exposure of the cells to inactivated IBV. The results indicate that splenocytes from chickens receiving oral bacteria, secreting LTB-N fusion protein, have significant higher activity against IBV, suggesting the at least small percentage of the splenocytes were specifically activated by the viral antigens (FIG. 7).

### Example 4

### Construction and Study of LTB-S Fusion Protein

The entire sequence of the IBV-S1 subunit (excluding the first 17 amino acids, all to the S⅟S2 cleavage site) was fused to the LTB sequence through a glycine rich linker. E. coli strain overexpressing the chimeric gene show higher secretion rate compared to the LTB-N fusion proteins. The LTB-S1 fusion protein was collected from the medium by ultrafiltration and was used in an animal study in which chickens were vaccinated orally as described earlier, in five groups: 1. WT E. coli (negative control); 2. Combination of two live E. coli secreting the LTB-N 218-326 and LTB-N 29-160 domains; 3. E. coli bacterium secreting the LTB-S1 fusion protein; Three strains of live bacteria, as a mix of the above LTB-N and LTB-S1; and 5. Partially purified LTB-S1 protein that was collected from the medium. Blood was extracted for analysis of serums, analyzed by a commercial ELISA kit (IBV ELISA; Biochek, Ascot UK) for active immune response against IBV. Data was positioned in the proper equation (instruction manual) for a positive cut-off number of 0.2. As shown in FIG. 8, all groups were found to be (e.g., tested) positive for IBV, with stronger reaction in the mix group (both N+S domain) and with strongest reaction for the LTB-S1 protein, given orally (50 µg/bird) as a purified protein (without the presence of bacterial cells).

### Example 5

### Vaccination Study of Chicken Against IBV

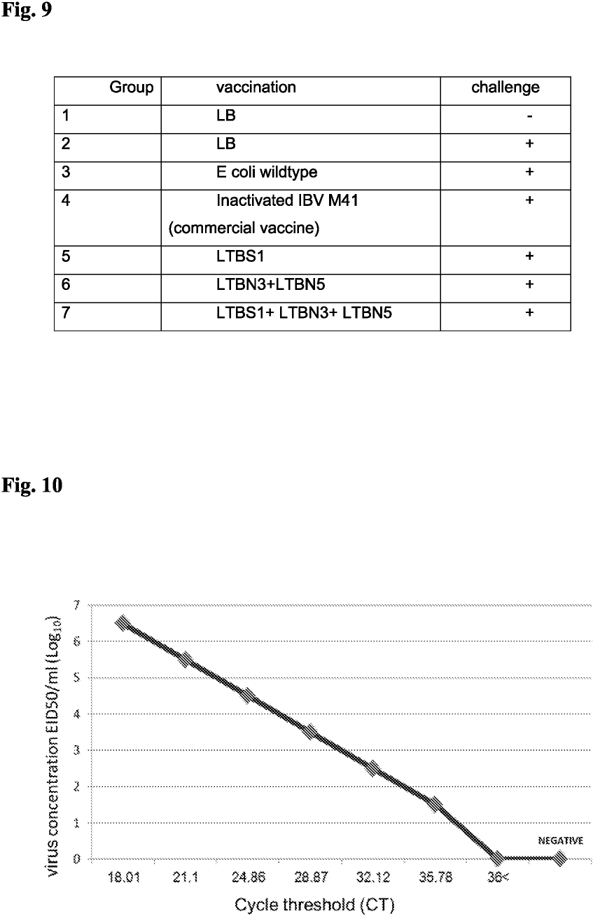

One (1) month old chickens were challenged with wild type IBV following oral intake of partially purified chimeric proteins LTB-S1, LTB-N3, and LTB-N5. The study contained 7 groups as described in FIG. 9. All potential vaccines and controls were administered twice, with two weeks interval, and then blood was collected from each chicken for analysis of serum antibodies. At the age of 60 days chickens were challenged with 200 µl of virulent IBV strain M41 (107.5 EID50/ml) administered equally into both the eyes and nostrils (50 µl each).

Swab samples were collected from the chicken’s trachea and cloaca at days 3, 6 and 10 post challenge, and then analyzed for presence of IBV (M41) by RT-PCR. Samples were quantified according to calibration curve displayed in FIG. 10.

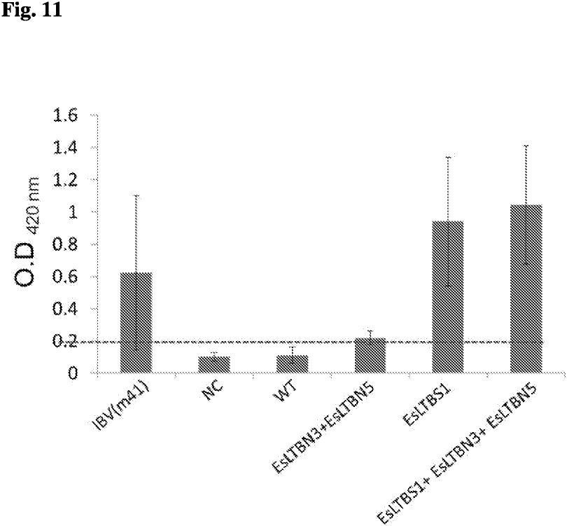

As shown in FIG. 11, oral administration of all tested proteins and their combinations, resulted in the presence of corresponding antibodies with strongest reaction in the mix (both N+S domains) and LTB-S1 groups.

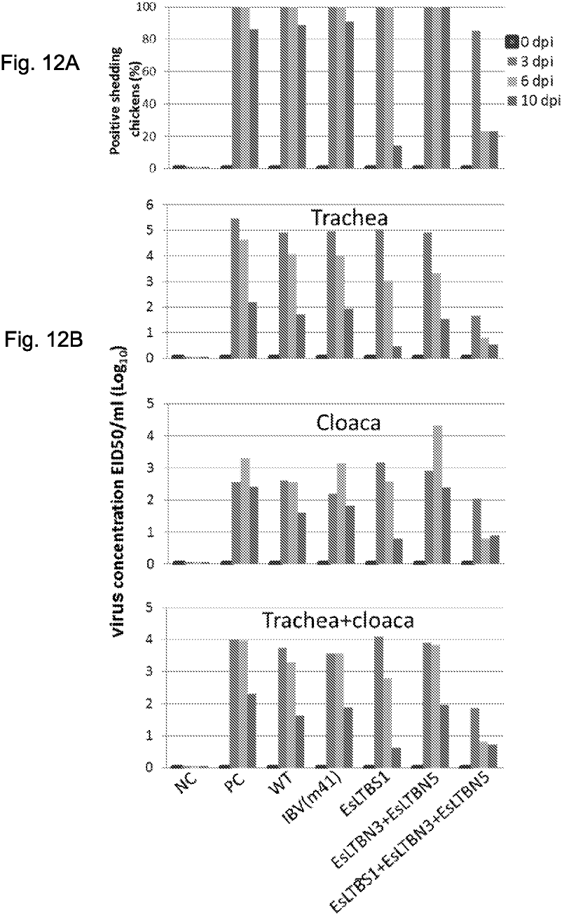

IBV shedding 3 days post challenge was detected in all of the chickens from groups 2-6 while shedding was detected in only 85% of chickens in group 7 vaccinated with LTBS1+ LTBN3+ LTBN5 (FIG. 12A). Quantification of both tracheal and cloacal swab samples showed a 1.9-fold decrease in virus EID50/ml levels measured in group 7 as compared to the WT group (group 3) at day 3 (FIG. 12B, bottom graph). At day 6 post challenge, only 23% of chickens in group 7 remained positively shedding while no decrease was evident for any other group (FIG. 12A). Quantified virus levels in the trachea at day 6 had decreased in all groups as compared to day 3. As compared to the WT control (group 2), the group vaccinated with the commercial IBV (M41) (group 4) vaccine showed no significant difference in the virus levels measured in the trachea at day 6 while lower levels were shown in groups 5, 6 and 7 (1.03-, 0.72- and 3.26-fold change respectively; FIG. 12B, top graph). In cloacal swabs tested at day 6, virus levels had dropped only in groups 5 and 7 (0.59- and 1.26-fold, respectively) as compared to day 3 while remaining stable in the WT control (group 3) and increasing in groups 2, 4 and 6 (FIG. 12B, middle graph). Accordingly, significant decrease in virus levels quantified from both tracheal and cloacal swab samples was also shown in groups 5 and 7 (1.3- and 1.08-fold change) while other groups retained stable virus levels (FIG. 12B, bottom graph).

At day 10 post challenge no further decrease in positively shedding chickens was shown in group 7 while in group 5 vaccinated with EsLTBS1 84% of chickens were tested negative for IBV shedding (FIG. 12A). No significant decrease in the percent of positively shedding chickens was evident in groups 3, 4, and 6, as compared to the spontaneous clearance in shedding displayed by the unvaccinated control (group 2; FIG. 12A).

Quantified virus levels in the trachea at day 10 had decreased in all groups as compared to days 3 and 6. As compared to the WT control (group 2), the group vaccinated with the commercial IBV (M41; group 4) vaccine showed an increase in the virus levels measured in the trachea at day 10 while lower levels were shown in groups 5, 6, and 7 (1.25-, 0.18- and 1.16-fold difference, respectively; FIG. 12B, top graph). In cloacal swabs tested at day 10, virus levels had dropped in all groups except group 7 which retain stable levels as compared to day 6. Still, the lowest virus levels in cloacal samples were detected in groups 5 and 7 (0.79 and 0.89 EID50/ml, respectively; FIG. 12B, middle graph).

Accordingly, a significant decrease in virus levels quantified from both tracheal and cloacal swab samples was shown in all groups except group 7, which retained stable virus levels. Still, the lowest virus levels in those samples were detected in groups 5 and 7 (0.63 and 0.72 EID50/ml, respectively; FIG. 12B, bottom graph).

### Example 6

### Vaccination Study of Human Subjects Against Coronavirus With LTB Fusion Proteins

Human subjects are vaccinated using LTB being either fused or non-fused protein by oral administration, using either LTB and S1 as an active combination, or a different combination of LTB, S1, and LTB-N proteins as three active components, according to the following groups: (1) oral intake of LTB and S1; (2) oral intake of LTB, S1, and LTB-NN (N-terminal domain of nucleocapsid protein); (3) LTB, S1, and LTB-NC (C-terminal domain of nucleocapsid protein); (4) LTB, S1, LTB-NN, and LTB-NC; and (5) negative control based on E. coli lysate representing the expression vector proteins.

All subjects are administered twice, with two weeks interval, and then blood is collected from each subject for analysis of serum antibodies. The sera of each group are reacted with: (1) Total proteins of WT bacteria; (2) Partially purified LTB; (3) Partially purified N; and (4) Partially purified S1, and analyzed using both enzyme-linked immunosorbent assay (ELISA) and western blot, as described above. Reactive LTB-fusion proteins can be than further optimized so as to increase immunogenic activity and subsequently improve immunization.

### Example 7

### In Vivo Vaccination

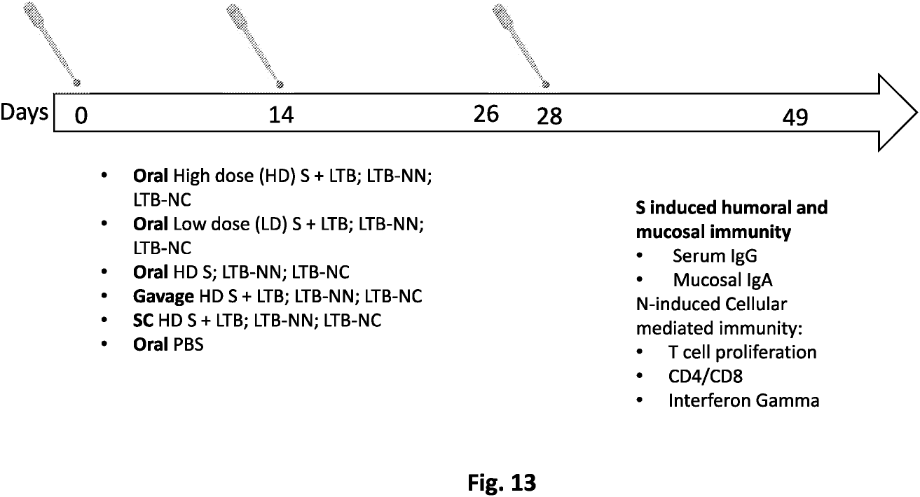

Mice were vaccinated with vaccines, as described in FIG. 13, either orally or by gavage.

Experimental groups were as follows:
(1) Oral High dose (HD) S (SEQ ID NO: 2 or SEQ ID NO: 24) + LTB (SEQ ID NO: 26); LTB (SEQ ID NO: 1)-NN (SEQ ID NO: 28); and LTB (SEQ ID NO: 1)-NC (SEQ ID NO: 30);
(2) Oral Low dose (LD) S (SEQ ID NO: 2 or SEQ ID NO: 24) + LTB (SEQ ID NO: 26); LTB (SEQ ID NO: 1)-NN (SEQ ID NO: 28); and LTB (SEQ ID NO: 1)-NC (SEQ ID NO: 30);
(3) Oral HD S (SEQ ID NO: 2 or SEQ ID NO: 24); LTB (SEQ ID NO: 1)-NN (SEQ ID NO: 28); and LTB (SEQ ID NO: 1)-NC (SEQ ID NO: 30);
(4) Gavage HD S (SEQ ID NO: 2 or SEQ ID NO: 24); LTB (SEQ ID NO: 1)-NN (SEQ ID NO: 28); and LTB (SEQ ID NO: 1)-NC (SEQ ID NO: 30); and
(5) Oral PBS.

Each mouse received 3 vaccine doses on day 0, 14, and 28. On day 26 blood was taken and evaluated for antibody levels after 2 vaccine doses. On day 49 blood and faeces were collected then the mice were sacrificed, and spleens were harvested and dissociated into single cell suspension. Enzyme-linked immunosorbent assay (ELISA) was used to evaluate the levels of serum IgG against Spike protein 1 (S1) in the sera and mucosal IgA antibodies in feces. Cellular mediated immunity against N protein was assessed on splenocytes by MTT proliferation assay with ALAMAR blue, and by ELISpot.

The titers of anti S1 serum IgG were measured using ELISA, as described hereinbelow.

Plates were coated with S1 over-night 4° C. then blocked with skim milk. Sera from each mouse were 2 folds diluted serially from 1:32 up to 1:65,536, then all serum dilutions were incubated in the plates for 1 hour at 37° C. Goat anti-mouse IgG -peroxidase was incubated in the plates for 1 hour at 37° C. followed by addition of OPD substrate and absorption reading at 450 nm. Between each step plates were washed 3 times with PBS+ 0.05% tween20.

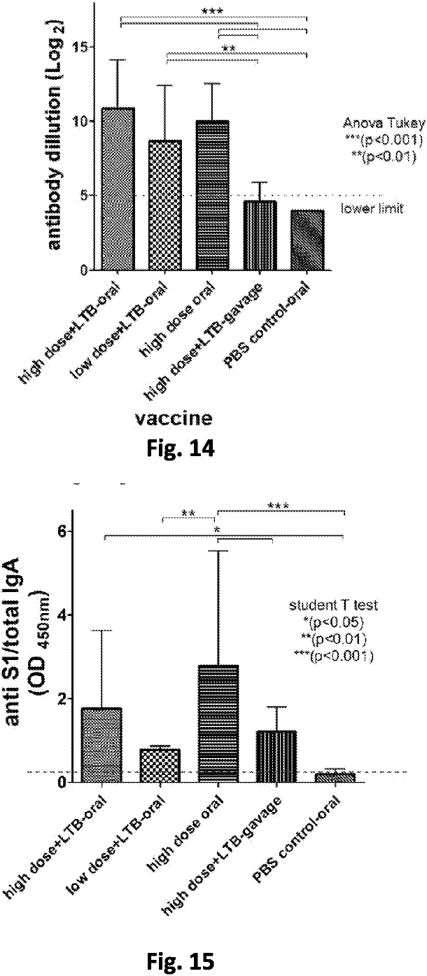

The results show that a significant elevation of IgG levels against S1 was found in sera of all oral vaccinations. In addition, oral route administration, provided significantly greater titers than gavage (FIG. 14).

The levels of anti S1 IgA in feces were measured using ELISA, as follows: Plates were coated with S1 over-night at 4° C. and then blocked with skim milk. Mouse faeces (4-5 pellets) directly taken from the colon and frozen were weighted and suspended in PBS in 1:1 volume (ml) to weight (mg). Fecal samples were diluted ×2 and then incubated in the plates for 1 hour at 37° C. Goat anti mouse IgA-peroxidase, and substrate were added, and washing steps performed as described above.

The results show a significant increase in secretory anti-S1 IgA levels in the mouse feces, which represent mucosal immune response in mice vaccinated with any vaccine combination as compared to mice to which PBS was administered (FIG. 15).

The levels of anti S1 serum IgG after a second dose vs a third dose of vaccination, as measured by ELISA.

Plates were coated and further assayed as described above (for serum anti S1 IgG). In order to compare the difference in antibody levels between the second and third vaccination doses, each individual OD value was normalized with the average value received from the PBS-vaccinated (control) group.

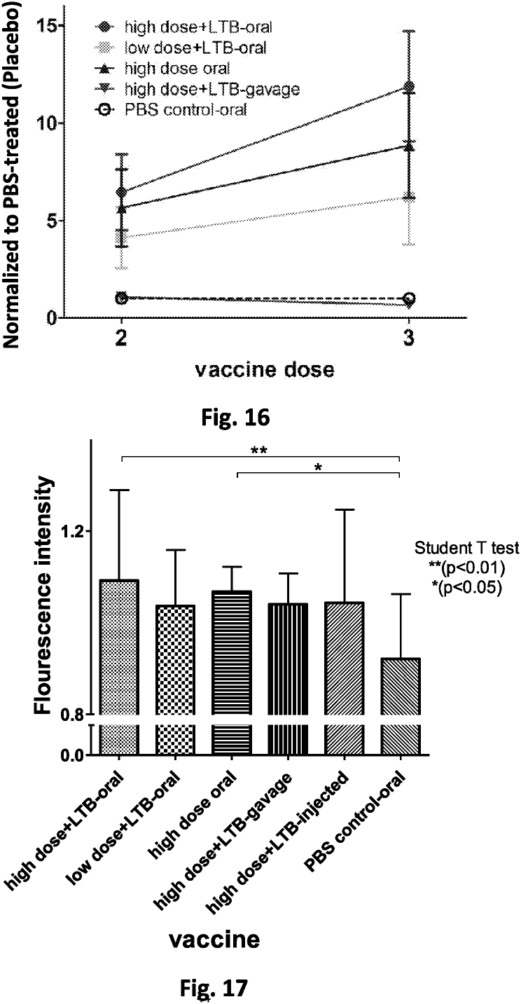

The results show that significant levels of serum IgG were observed after a second dose of an orally provided vaccine (compared to the control; FIG. 16). Following a third administration of the oral vaccine, the levels of serum IgG observed were further increased compared to the second dose provided (FIG. 16).

The inventors further examined splenocyte cell proliferation using the MTT proliferation assay.

Splenocytes from each mouse were seeded in a 24-well plate at a concentration of 5 × 10⁶ cells/ml and allowed to settle for 1 hour, then mixed 1:1 volume with media containing 20 µl/ml of Nucleocapsid protein (N, no protein addition was used as control). The cells and inducer mix were incubated over-night at 37° C. and 5% CO₂, then 100 µl samples were taken from each well and seeded in black optic-bottom 96 wells. Plates were incubated for additional 24 hours, 10 µl ALAMAR blue were added and incubated for additional 4 hours. Fluorescence was measured (560 nm/590 nm) to quantify cells in each well. Reading of cells incubated with the N protein were divided with the readings of cells without an additional protein for normalization (FIG. 17).

For ELISpot assay, splenocytes from each mouse were seeded in 24 well plate at a concentration of 5 × 10⁶ cells/ml and allowed to settle for 1 hour, then mixed 1:1 volume with media containing 20 µl of the N protein (no protein addition was used as control). The cells and inducer mix were incubated over-night at 37° C. and 5% CO₂, then 100 µl samples were taken from each well and seeded in anti-interferon gamma (INF) coated ELISpot plates. Plates were incubated for additional 17 hours and then washed. Plates were stained with anti INF gamma-peroxidase antibodies and substrate. Spots numbers were counted with ELISpot reader. The number of spots received from cells incubated without protein was subtracted from the number received from cells incubated with the N protein.

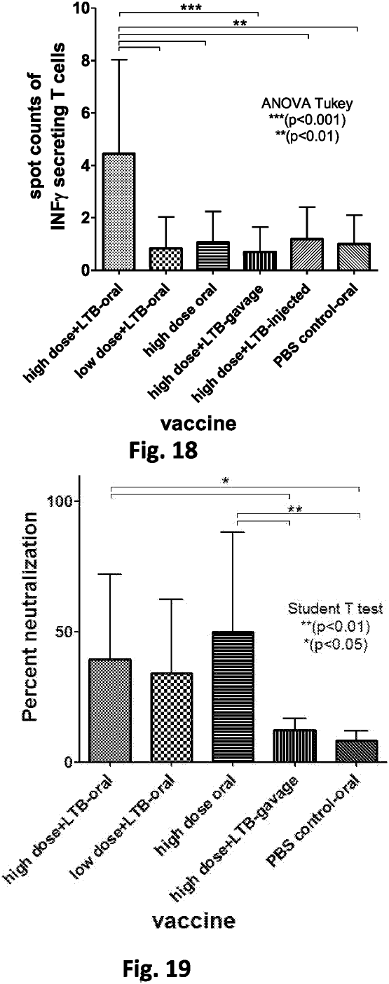

The results show that: (i) a significantly greater number of splenocytes was observed after induction with the N-protein using the proliferation assay (FIG. 17), and (ii) a significantly greater number of specific INF gamma secreting T cells was observed using the ELISPOT assay (FIG. 18). Further, the inventors observed no shift in CD8/CD4 population ratio, when assayed using fluorescence-activated cell sorting (FACS; data not shown).

Sera were further tested neutralization with cPass kit. Sera were diluted 10-fold and tested for the ability to inhibit the binding of RBD protein to the ACE2 receptor.

Orally administered vaccine resulted in neutralizing antibodies as compared to vaccination by Gavage and the vaccination control (FIG. 19).

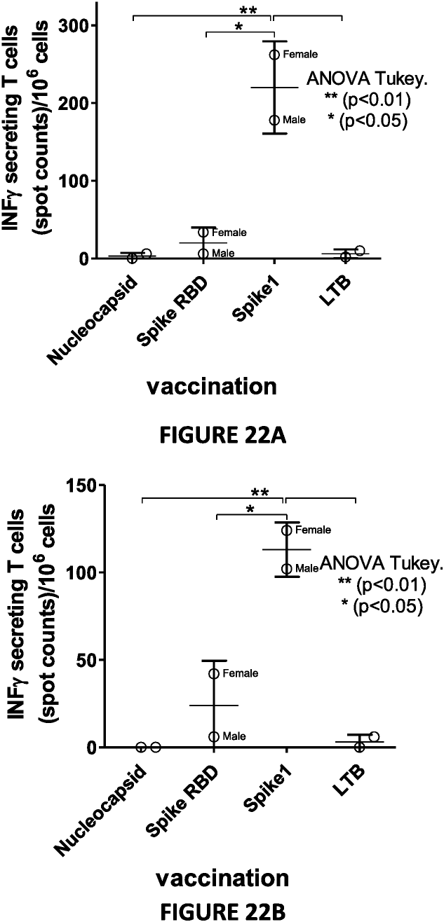

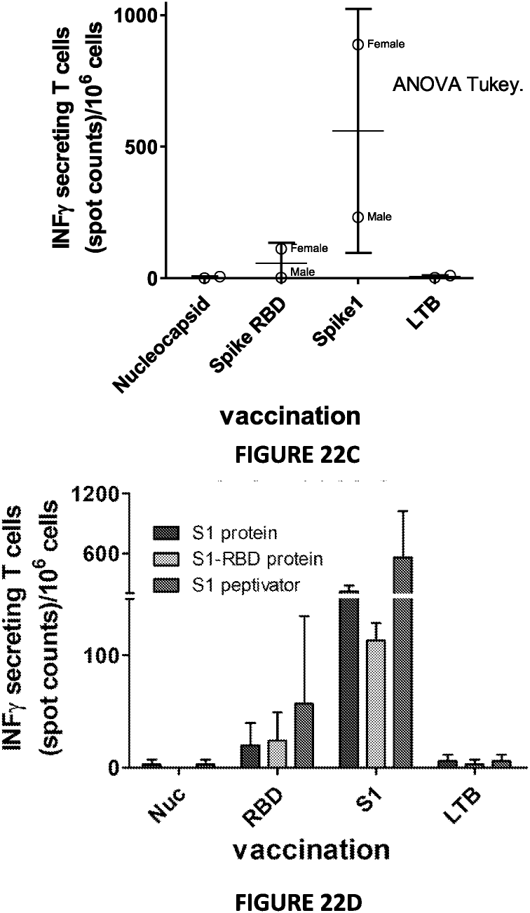

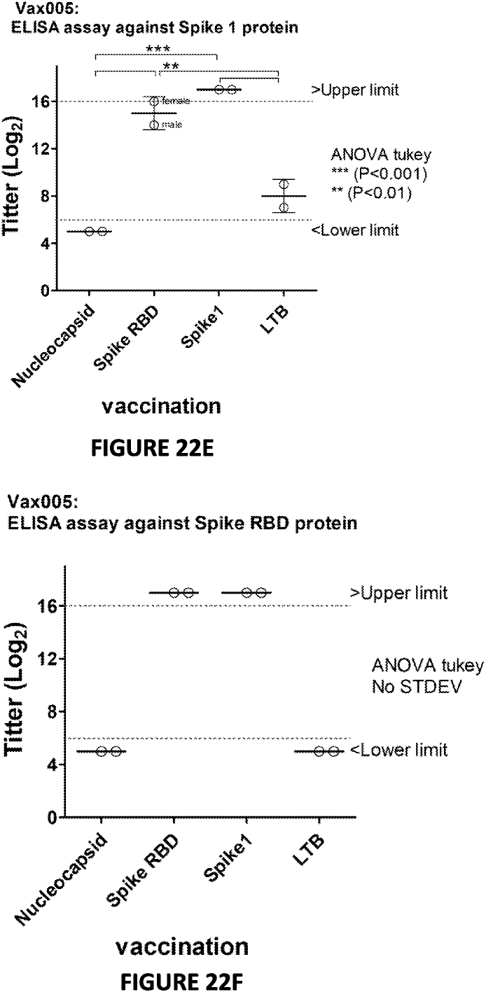

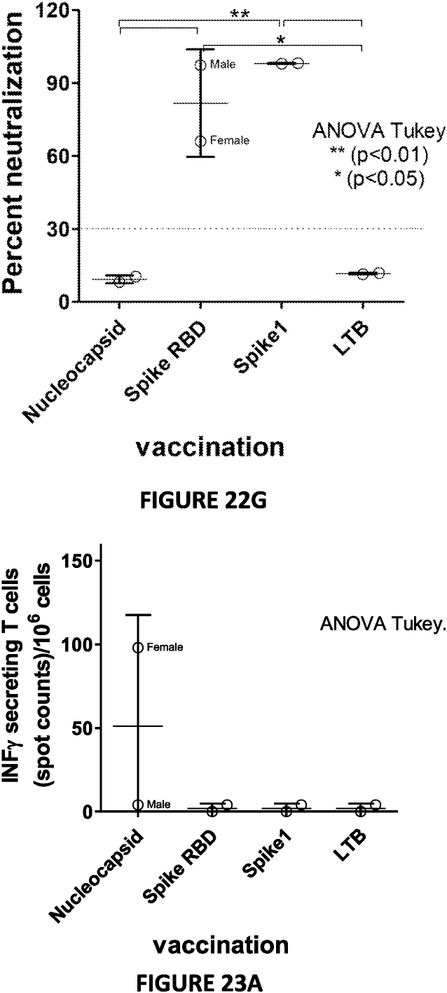

High titers of serum IgG were observed in rats vaccinated by injection of S1 or S1-RBD (FIGS. 22E-22F). The raised antibodies were further shown in an in vitro assay to be able to neutralize ACE2 - RBD interaction (FIG. 22G). On isolated splenocytes of these rats, S1 peptide pool was shown to be highly efficient in T cell activation (FIGS. 22C-22D). Also, splenocytes from rats vaccinated with either S1 protein or S1-RBD protein demonstrated greater numbers of specifically activated T cells compared to the splenocytes from rats vaccinated with N or LTB proteins (controls, FIGS. 22A-22B, respectively).

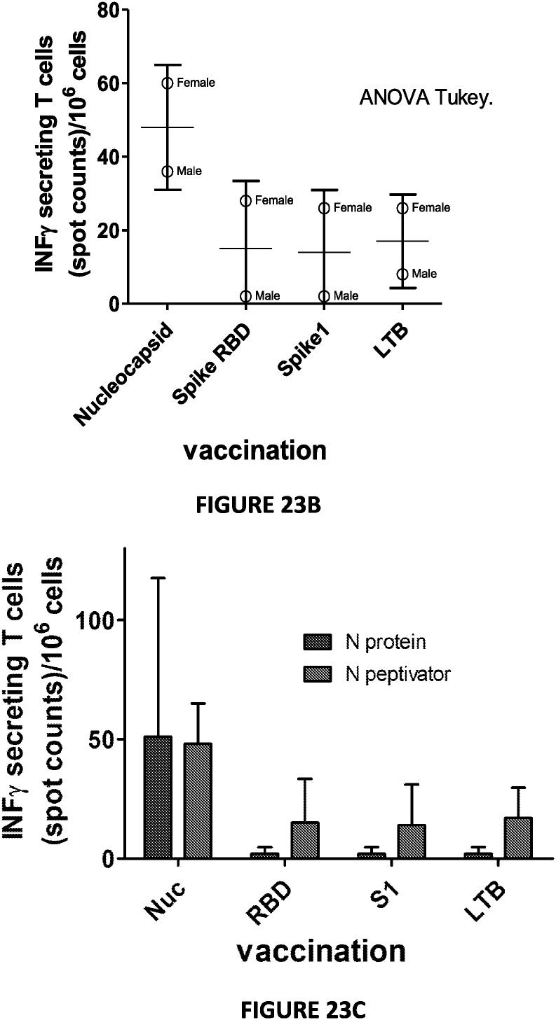

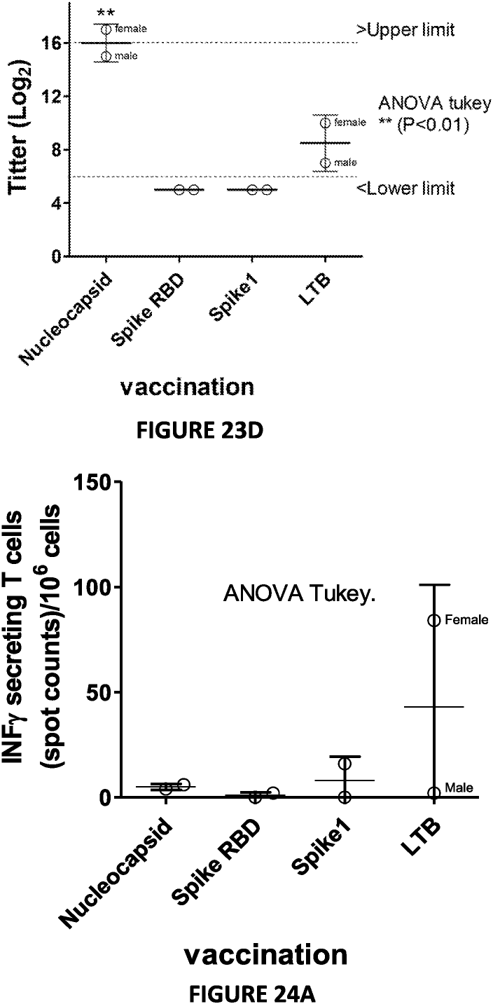

High titers of serum IgG were observed in vaccinations including the nucleocapsid protein (N; FIG. 23D). Splenocytes from rats vaccinated with the N protein showed elevated numbers of specifically activated T cells secreting interferon gamma after induction with N protein and N peptide pool (Peptivator™; FIGS. 23A-23C).

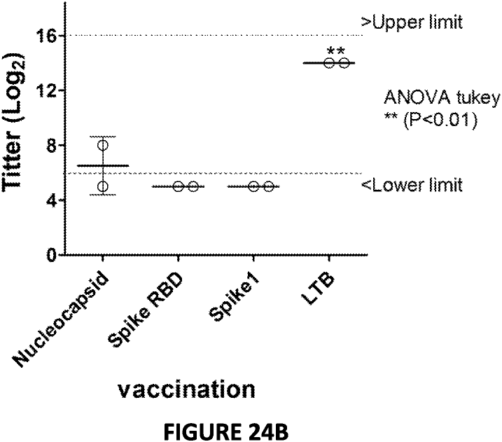

Splenocytes from rats vaccinated with LTB protein demonstrated relatively high numbers of specifically activated T cells secreting interferon gamma after induction with the LTB protein (FIG. 24A). High titters of serum IgG were observed in vaccinations including the LTB protein (FIG. 24B).

### Example 8

### Structure and Sequence Comparison Table the Spike Protein of SARS-CoV-2 and SARS-CoV-1, MERS and IBV

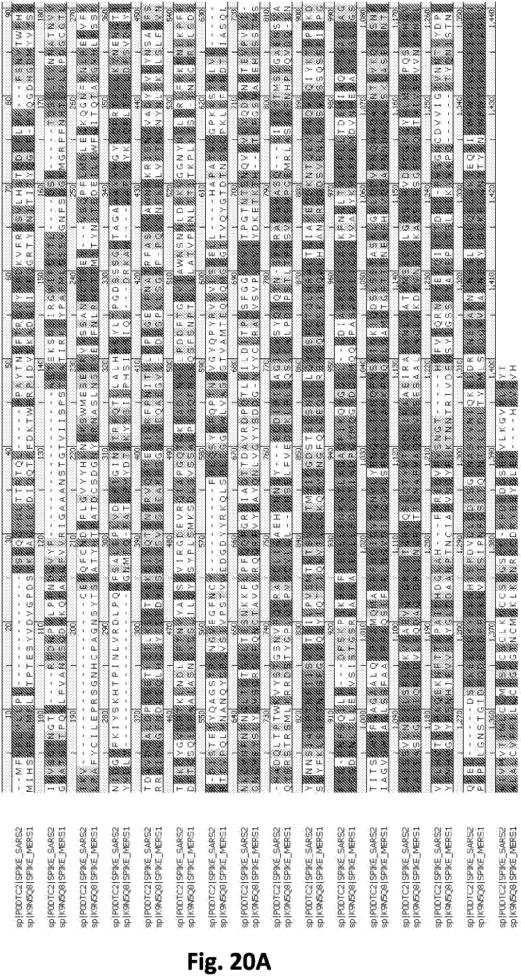

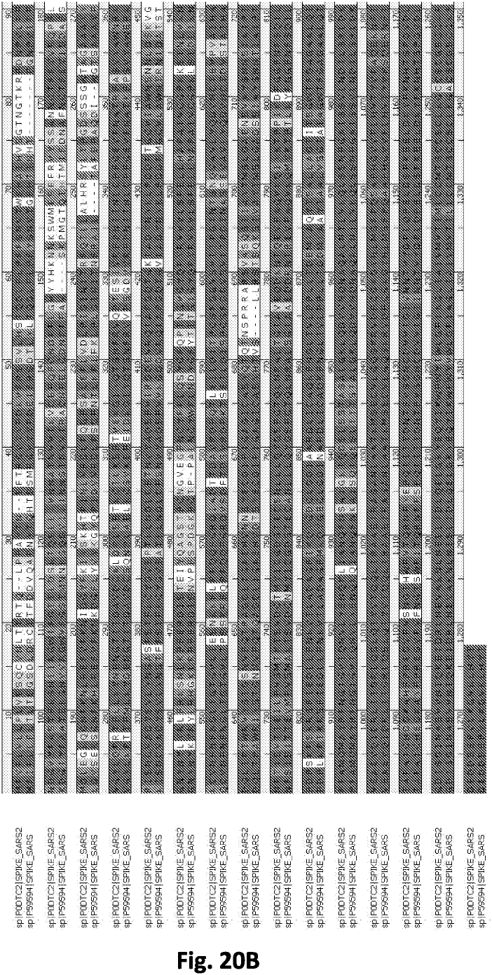

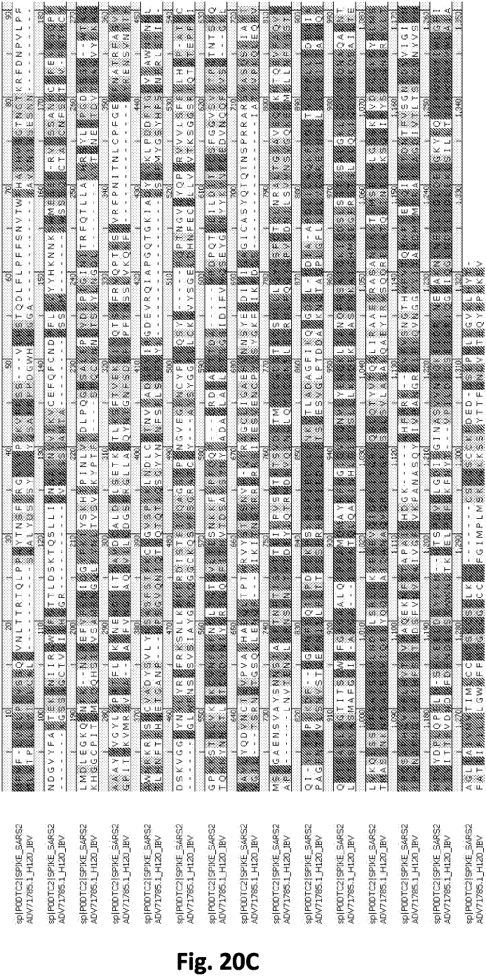

Sequence alignment was performed between the spike protein of SARS-CoV-2 and IBV, SARS-CoV1 and MERS. The alignments are presented under FIGS. 20A-20C and a summary of the identity and similarity is shown under Table 1.

TABLE 1

| Alignment | Identity | Similarity |
|---|---|---|
| Cov-2 vs. IBV | 24.5% | 43.3% |
| Cov-2 vs. MERS | 29.3% | 49.4% |
| Cov-2 vs. SARS-CoV1 | 76% | 88.4% |

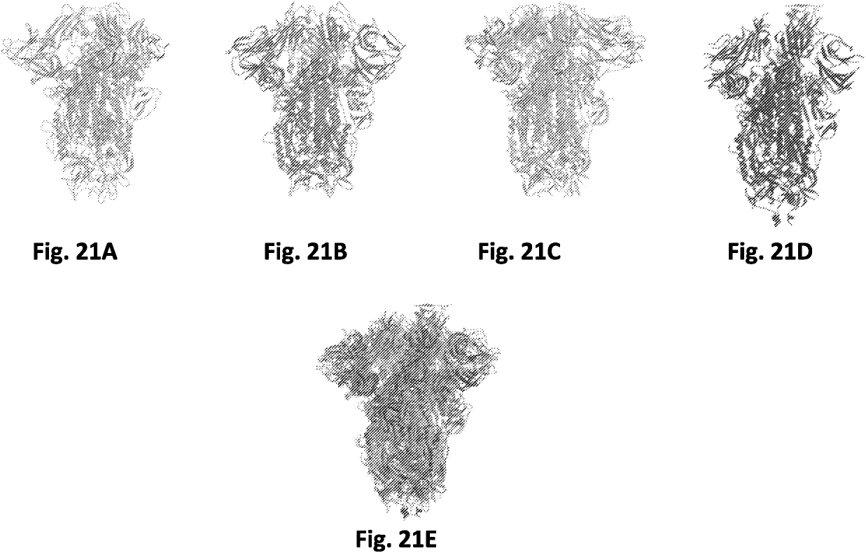

Structural comparison analysis between the spike protein of SARS-CoV-2 and IBV, SARS-CoV1 and MERS, was made. The results are shown under FIG. 21 and are also presented under Table 2:

TABLE 2

| CoV-2 | structure 2 | origin | RMSD Ca (trimer) |
|---|---|---|---|
| 6VXX | 6CV0 | IBV | 1.87 |
| 6VXX | 5X58 | SARS (CoV1) | 1.54 |
| 6VXX | 6Q04 | MERS | 1.85 |

The Root mean square distance is in Angstrom units (Å)

As seen in FIG. 21 the viral spike proteins of IBV, MERS, SARS-CoV1 and SARS-CoV-2 are structurally similar determined according to the level of the root mean square distancing (RMSD).

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A composition comprising:
a. a heat labile toxin subunit B (LTB) polypeptide comprising the sequence:
MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKN (SEQ ID NO: 1)
or an analog thereof having at least 80% sequence identity to said LTB; and
b. a plurality of immunogenic polypeptides;
wherein said plurality of immunogenic polypeptides comprises at least two viral peptides or any analogs thereof having at least 80% sequence identity to said at least two viral peptides.

2. The composition of claim 1, comprising a first viral peptide, and said LTB conjugated to at least a second viral peptide, thereby forming a chimeric polypeptide, and optionally wherein said LTB polypeptide comprises a plurality of LTB polypeptides.

3. (canceled)

4. The composition of claim 3, wherein said plurality of LTB polypeptides comprises: (i) at least a first LTB polypeptide being a non-conjugated LTB; (ii) at least a second LTB polypeptide conjugated to at least one peptide of said plurality of immunogenic polypeptides, thereby forming a chimeric polypeptide; or (iii) any combination of (i) and (ii), and optionally wherein said plurality of LTB polypeptides comprises at least a first LTB polypeptide being a non-conjugated LTB and at least a second LTB polypeptide conjugated to at least one peptide of said plurality of immunogenic polypeptides, thereby forming a chimeric polypeptide.

5. (canceled)

6. The composition of claim 1, wherein said at least two viral peptides comprise (a) a viral spike protein; and (b) a viral nucleocapsid protein, wherein said at least two viral peptides comprise the full length amino acid sequence or a partial amino acid sequence of said viral spike protein and of said viral nucleocapsid protein, or an analog of any one of said spike protein and of said nucleocapsid protein, having at least 80% sequence identity to any one of said spike protein and said nucleocapsid protein, optionally wherein: (i) wherein said spike protein comprises the amino acid sequence:
EFITNLCPFGEVFNATRFASVYAWNRKRISNCVADYSVLYNSASFSTFKC
YGVSPTKLNDLCFTNVYADSFVIRGDEVRQIAPGQTGKIADYNYKLPDDF
TGCVIAWNSNNLDSKVGGNYNYLYRLFRKSNLKPFERDISTEIYOAGSTP
CNGVEGFNCYFPLOSYGFOPTNGVGYQPYRVVVLSFELLHAPATVCGPKK
STNLVKNKXVNFNFNGLTGT (SEQ ID NO: 24),
wherein X is cysteine or alanine; or analog having at least 80% sequence identity to SEQ ID NO: 24; (ii) said spike protein comprises the amino acid sequence: (a)
VNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANN
CTFEYVSOPFLMDLEGKQGNFKNLREFVFKNIDGYFKIYSKHTPINLVRD
LPQGFSALEPLVDLPIGINITRFQTLLALHRSYLTPGDSSSGWTAGAAAY
YVGYLQPRTFLLKYNENGTITDAVDCALDPLSETKCTLKSFTVEKGIYQT
SNFRVQPTESIVRFPNITNLCPFGEVFNATRFASVYAWNRKRISNCVADY
SVLYNSASFSTFKCYGVSPTKLNDLCFTNVYADSFVIRGDEVROIAPGOT
GKIADYNYKLPDDFTGCVIAWNSNNLDSKVGGNYNYLYRLFRKSNLKPFE
RDISTEIYOAGSTPCNGVEGFNCYFPLQSYGFOPTNGVGYOPYRVVVLSF
ELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYODV
NCTEVPVAIHADOLTPTWRVYSTGSNVFOTRAGCLIGAEHVNNSYECDIP
IGAGICASYQTQTNSPRRAR (SEQ ID NO: 2);
(b)
ALYDSSSYVYYYQSAFRPPDGWHLHGGAYAVVNISSESNNAGSSSGCTVG
IIHGGRVVNASSIAMTAPSSGMAWSSSQFCTAYCNFSDTTVFVTHCYKHG
GCPITGMLQQHSIRVSAMKNGQLFYNLTVSVAKYPTFKSFQCVNNLTSVY
LNGDLVYTSNETTDVTSAGVYFKAGGPITYKVMREVRALAYFVNGTAQDV
ILCDGSPRGLLACQYNTGNFSDGFYPFTNSSLVKQKFIVYRENSVNTTFT
LHNFTFHNETGANPNPSGVQNIQTYQTQTAQSGYYNFNFSFLSSFVYKES
NFMYGSYHPSCNFRLETINNGLWFNSLSVSIAYGPLQGGCKQSVFSGRAT
CCYAYSYGGPLLCKGVYSGELDHNFECGLLVYVTKSGGSRIQTATEPPVI
TQHNYNNITLNTCVDYNIYGRTGQGFITNVTDSAVSYNYLADAGLAILDT
SGSIDIFVVQSEYGLNYYKVNPCEDVNQQFVVSGGKLVGILTSRNETGSQ
LLENQFYIKITNGTRRFRR (SEQ ID NO: 3);
or (c) any analog having at least 80% sequence identity to SEQ ID Nos.: 2 or 3; or (iii) said nucleocapsid protein comprises the amino acid sequence: (a)
NNTASWFTALTQHGKEDLKFPRGQGVPINTNSSPDDQIGYYRRATRRIRG
GDGKMKDLSPRWYFYYLGTGPEAGLPYGANKDGIIWVATEGALNTPKDHI
GTRNPANNAAIVLQLPQGTTLPKGFYAEGS (SEQ ID NO: 4);
(b)
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKT (SEQ IDNO: 5);
(c)
VGSSGNASWFOALKAKKLNSPPPKFEGSGVPDNENLKLSOOHGYWRRQAR
YKPGKGGKKSVPDAWYFYYTGTGPAADLNWGDSQDGIVWVSAKGADTKSR
SNQGTRDPDKFDQYPLRFSDGGPDGNFRWDFIPI (SEQ ID NO: 6);
(d)
KADEMAHRRYCKRTIPPGYKVDQVFGPRTKGKEGNFGDDKMNEEGIKDGR
VIAMLNLVPSSHACLFGSRVTPKLQPDGLHLRFEFTTVVSRDDPOFDNYV
KICDOCVDG (SEQ IDNO: 7);
or (e) any analog having at least 80% sequence identity to SEQ ID NO: 4, SEQ ID NO: 5, SEQ ID NO: 6, or SEQ ID NO: 7.

7-9. (canceled)

10. The composition of claim 2, wherein said conjugated is via a peptide linker comprising an amino acid sequence of 2 to 10 amino acids, optionally wherein said linker comprises 3 to 7 amino acids, optionally wherein said linker comprises Serine and Glycine amino acid residues, and optionally wherein said linker consists of Serine and Glycine amino acid residues.

11-13. (canceled)

14. The composition of claim 3, comprising any one of: (i) (a) an LTB polypeptide being a non-conjugated LTB; and said at least two viral peptides; (b) a first LTB polypeptide being a non-conjugated LTB; a first viral peptide; and a chimeric polypeptide comprising at least a second LTB polypeptide conjugated to at least a second viral peptide; (c) a first chimeric polypeptide comprising a first LTB polypeptide conjugated to at least a first viral peptide and a second chimeric polypeptide comprising at least a second LTB polypeptide conjugated to at least a second viral peptide; (d) a first viral peptide; and a chimeric polypeptide comprising an LTB polypeptide conjugated to at least a second viral peptide; or (e) any combination of (a) to (d); (ii) wherein said chimeric polypeptide comprises the sequence of:
(a)
MGNKVKCYVLFTALLSSLYAHGAPQTITELCSEYRNTQIYTINDKILSYT
ESMAGKREMVIITFKSGETFOVEVPGSOHIDSQKKAIERMKDTLRITYLT
ETKIDKLCVWNNKTPNSIAAISMKNGGSGGVNLTTRTQLPPAYTNSFTRG
VYYPDKVFRSSVLHSTQDLFLPFFSNVTWFHAIHVSGTNGTKRFDNPVLP
FNDGVYFASTEKSNIIRGWIFGTTLDSKTQSLLIVNNATNVVIKVCEFQF
CNDPFLGVYYHKNNKSWMESEFRVYSSANNCTFEYVSQPFLMDLEGKQGN
FKNLREFVFKNIDGYFKIYSKHTPINLVRDLPQGFSALEPLVDLPIGINI
TRFQTLLALHRSYLTPGDSSSGWTAGAAAYYVGYLQPRTFLLKYNENGTI
TDAVDCALDPLSETKCTLKSFTVEKGIYQTSNFRVQPTESIVRFPNITNL
CPFGEVFNATRFASVYAWNRKRISNCVADYSVLYNSASFSTFKCYGVSPT
KLNDLCFTNVYADSFVIRGDEVRQIAPGQTGKIADYNYKLPDDFTGCVIA
WNSNNLDSKVGGNYNYLYRLFRKSNLKPFERDISTEIYQAGSTPCNGVEG
FNCYFPLOSYGFOPTNGVGYOPYRVVVLSFELLHAPATVCGPKKSTNLVK
NKCVNFNFNGLTGTGVLTESNKKFLPFQQFGRDIADTTDAVRDPQTLEIL
DITPCSFGGVSVITPGTNTSNQVAVLYQDVNCTEVPVAIHADQLTPTWRV
YSTGSNVFQTRAGCLIGAEHVNNSYECDIPIGAGICASYQTQTNSPRRAR
(SEQ ID NO: 8);
or (b)
MGNKVKCYVLFTALLSSLYAHGAPOTITELCSEYRNTQIYTINDKILSYT
ESMAGKREMVIITFKSGETFOVEVPGSOHIDSQKKAIERMKDTLRITYLT
ETKIDKLCVWNNKTPNSIAAISMKNEFITNLCPFGEVFNATRFASVYAWN
RKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYR
LFRKSNLKPFERDISTEIYOAGSTPCNGVEGFNCYFPLOSYGFQPTNGVG
YOPYRVVVLSFELLHAPATVCGPKKSTNLVKNKXVNFNFNGLTGT (SEQ
ID NO: 24),
wherein X is cysteine or alanine; (iii) wherein said chimeric polypeptide comprises the sequence of:
MGNKVKCYVLFTALLSSLYAHGAPOTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGSGGNNTASWFTALTQHGKEDLKF
PRGQGVPINTNSSPDDQIGYYRRATRRIRGGDGKMKDLSPRWYFYYLGTG
PEAGLPYGANKDGIIWVATEGALNTPKDHIGTRNPANNAAIVLQLPQGTT
LPKGFYAEGS (SEQ ID NO: 9);
(iv) wherein said chimeric polypeptide comprises the sequence of:
MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGSGGALYDSSSYVYYYQSAFRPPD
GWHLHGGAYAVVNISSESNNAGSSSGCTVGIIHGGRVVNASSIAMTAPSS
GMAWSSSQFCTAYCNFSDTTVFVTHCYKHGGCPITGMLQQHSIRVSAMKN
GOLFYNLTVSVAKYPTFKSFOCVNNLTSVYLNGDLVYTSNETTDVTSAGV
YFKAGGPITYKVMREVRALAYFVNGTAQDVILCDGSPRGLLACQYNTGNF
SDGFYPFTNSSLVKQKFIVYRENSVNTTFTLHNFTFHNETGANPNPSGVQ
NIQTYQTQTAQSGYYNFNFSFLSSFVYKESNFMYGSYHPSCNFRLETINN
GLWFNSLSVSIAYGPLOGGCKOSVFSGRATCCYAYSYGGPLLCKGVYSGE
LDHNFECGLLVYVTKSGGSRIOTATEPPVITQHNYNNITLNTCVDYNIYG
RTGQGFITNVTDSAVSYNYLADAGLAILDTSGSIDIFVVQSEYGLNYYKV
NPCEDVNQQFVVSGGKLVGILTSRNETGSQLLENQFYIKITNGTRRFRR
(SEQ ID NO: 11);
(v) wherein said chimeric polypeptide comprises the sequence of:
MGNKVKCYVLFTALLSSLYAHGAPOTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGSGGVGSSGNASWFQALKAKKLNS
PPPKFEGSGVPDNENLKLSQQHGYWRRQARYKPGKGGKKSVPDAWYFYYT
GTGPAADLNWGDSQDGIVWVSAKGADTKSRSNQGTRDPDKFDQYPLRFSD
GGPDGNFRWDFIPI (SEQ ID NO: 12);
and (vi) wherein said chimeric polypeptide comprises the sequence of:
MGNKVKCYVLFTALLSSLYAHGAPQTITELCSEYRNTQIYTINDKILSYT
ESMAGKREMVIITFKSGETFQVEVPGSQHIDSQKKAIERMKDTLRITYLT
ETKIDKLCVWNNKTPNSIAAISMKNGGSGGKADEMAHRRYCKRTIPPGYK
VDQVFGPRTKGKEGNFGDDKMNEEGIKDGRVIAMLNLVPSSHACLFGSRV
TPKLQPDGLHLRFEFTTVVSRDDPQFDNYVKICDQCVDG
(SEQ ID NO: 13)
.

15-20. (canceled)

21. The composition of claim 1, wherein said at least two viral peptides comprise at least one immunogenic antigen of a pathogenic virus, optionally wherein said pathogenic virus is an animal pathogen, optionally wherein said animal is selected from the group consisting of: a mammal, an avian, and a fish, optionally wherein said animal is a human subject, optionally wherein said pathogenic virus comprises a Coronavirus, optionally wherein said Coronavirus comprises any one of: the Wuhan human Corona 2020 (SARS-CoV-2), SARS-CoV, or MERS-CoV.

22-26. (canceled)

27. The composition of claim 21, wherein said pathogenic virus is an avian pathogenic virus and is selected from the group consisting of: Infectious bursal disease virus (IBDV), Infectious bronchitis virus (IBV), Reovirus, Influenza virus, Chicken anemia virus (CAV), Newcastle disease virus (NDV), Marek’s disease virus (MDV), Egg drop syndrome (EDS) avian adenovirus, and hemorrhagic enteritis virus (HEV), optionally wherein said at least one immunogenic antigen of IBDV is VP2; of IBV is S1, N, or combination thereof; of Reovirus is Sigma C; of Influenza virus is HA; of CAV is VP1 or VP2; of NDV is HN or F; of EDS is KNOB; and of HEV is KNOB.

28. (canceled)

29. A pharmaceutical composition comprising the composition of claim 1, and a pharmaceutically acceptable carrier, optionally being formulated for an administration to a subject via a route selected from the group consisting of: oral, transdermal, anal, nasal, topical, and in-ovo.

30-31. (canceled)

32. The pharmaceutical composition of claim 29, wherein said subject is infected with or at increased risk of infection of: Coronavirus, SARS-CoV-2, SARS-CoV, MERS-CoV, IBV, IBDV, Reovirus, Influenza virus, CAV, NDV, MDV, EDS avian adenovirus, and HEV, and optionally wherein said subject is selected from the group consisting of: a human, an avian, and a fish.

33. (canceled)

34. A polynucleotide molecule encoding the chimeric polypeptide of the composition of claim 2.

35. An expression vector comprising the polynucleotide molecule of claim 34.

36. A cell comprising the expression vector of claim 35, optionally wherein said cell is a naive cell or a recombinant cell.

37. (canceled)

38. The cell of claim 36 , further lacking one or more extra-cellular proteases.

39. The cell of claim 36, selected form the group consisting of: a bacterial cell, a plant cell, a mammalian cell, an insect cell, and a yeast cell.

40. The cell of claim 36, wherein said cell is an E. coli cell.

41. The cell of claim 36,further comprising a general secretory pathway (GSP) operon encoding a type II secretion system; and optionally lacking a polynucleotide comprising a gene encoding transcription factor histone-like nucleoid-structuring protein (HNS).

42. The cell of claim 36, being an E. coli cell derived from a naïve (non-recombinant) E. coli K12 or ER2566.

43. A composition comprising the cell of claim 36, and an acceptable carrier.

44. A method for treating or preventing a viral infection in a subject in need thereof, comprising administering to said subject a therapeutically effective amount of: (i) the composition of claim 1 , thereby treating or preventing a viral infection in the subject, optionally wherein said administering is by a route selected from the group consisting of: oral, transdermal, anal, nasal, topical, and in-ovo.

45. (canceled)
